(19) United States
(12) Patent Application Publication
(10) Pub. No.: US 2024/0033737 A1
(43) Pub. Date: Feb. 1, 2024

(54) DEVICES AND METHODS FOR CONTINUOUS DIELECTROPHORESIS CELL SORTING TO ISOLATE DIFFERENT POPULATIONS OF CELLS, AND APPLICATIONS THEREOF

(71) Applicant: The Regents of the University of California, Oakland, CA (US)

(72) Inventors: Lisa A. Flanagan, Irvine, CA (US); Alan Y. L. Jiang, Irvine, CA (US); Jaclyn Nicole Hanamoto, Irvine, CA (US); Clarissa C. Ro, Irvine, CA (US)

(21) Appl. No.: 18/226,739

(22) Filed: Jul. 26, 2023

Related U.S. Application Data
(60) Provisional application No. 63/392,488, filed on Jul. 26, 2022.

(51) Int. Cl. B01L 3/00

(52) U.S. Cl. CPC B01L 3/502761; B01L 3/50273; B01L 2400/0424; B01L 2200/0652; B01L 2300/0883; B01L 2300/0645

(57) ABSTRACT

The disclosure provides devices, methods and systems for continuous dielectrophoresis cell sorting to isolate different populations of cells, and applications thereof.

### CROSS REFERENCE TO RELATED APPLICATIONS

This application claims priority under 35 U.S.C. § 119 from Provisional Application Ser. No. 63/392,488 filed Jul. 26, 2022, the disclosure of which is incorporated herein by reference.

### STATEMENT OF GOVERNMENT SUPPORT

This invention was made with Government support under Grant No. R21NS111303, awarded by the National Institutes of Health and Grant No. IOS-1254060 awarded by the National Science Foundation. The Government has certain rights in the invention

### TECHNICAL FIELD

The disclosure provides devices, methods and systems for continuous dielectrophoresis cell sorting to isolate different populations of cells, and applications thereof.

### BACKGROUND

Glioblastoma (GBM) is one of the most complex, deadly, and treatment-resistant cancer that occurs in the brain or spinal cord. It accounts for 48% of all primary malignant brain tumors. Due to the fast-proliferating nature of these cells, patients diagnosed with this disease have a median survival of only 15-months. According to the national brain tumor society, there are more than 13,000 new cases in the United States each year. Temozolomide (TMZ), an orally delivered alkylating agent, is an FDA approved first-line treatment for glioblastoma. When orally administered, the prodrug temozolomide is readily absorbed in the small intestine, with good penetration of the blood-brain barrier due to its small size (194 Da). Once taken up by cells, TMZ is hydrolyzed into its active form, a potent methylating agent. The TMZ induced methylation of DNA triggers apoptosis due to mismatch repair. Unfortunately, patients that respond well to initial TMZ treatment, can have tumor regrowth that is refractory to TMZ treatment. GBM acquired resistance to TMZ is a major limitation for effective treatment of GBM.

The rapid identification and enrichment of TMZ-resistant cells from resected patient tumors could lead to a better understanding of their molecular characteristics, potential mechanisms of resistance, and promote development of more effective treatments. Conventional cell sorting systems, such as fluorescent-activated cell sorting (FACS) and magnetic-activated cell sorting (MACS), are label-based sorting methods that rely on well-defined biomarkers that are often not available for many important cell types, including drug-resistant GBM cells. Therefore, there is a need for developing better cell sorting techniques to screen for cancer cells that are resistant to chemotherapy agents.

### SUMMARY

The disclosure provides innovative devices, techniques and methods that utilize continuous dielectrophoresis (DEP) for cell sorting in order to isolate different populations of cells (e.g., drug resistant cancer cells v. non drug resistant cancer cells). DEP is an electrokinetic technique that can induce cell movement based on the innate dielectric properties of the cell, without using any labels. It was tested herein whether TMZ resistant GBM cells could be identified or enriched by comparing the DEP response of TMZ resistant and control cells within a frequency spectrum (see FIG. 6A). It was found herein that GBM cells that varied in TMZ resistance could be identified by membrane capacitance, since cells with high TMZ resistance had significantly lower membrane capacitance compared to cells with low TMZ resistance (see FIG. 6B). The midpoint membrane frequency, which predicts whether cells can be effectively sorted by DEP, was higher for TMZ resistant cells compared to controls (see FIG. 6C). To enrich for TMZ resistant GBM cells using DEP, new methods and devices were developed. For use with the new device, innovative buffers for DEP were developed for use in the methods disclosed herein. These new DEP buffers were optimized to maintain post sort acute and long-term cell viability (see FIG. 2). The optimized DEP buffer and the devices disclosed herein enabled the enrichment of TMZ-resistant cells. Moreover, post-sorting the TMZ-resistant cells remained viable after multiple passages and further retained TMZ-resistance. The methods and improved DEP devices disclosed herein take advantage of the intrinsic properties of cells, accordingly, the use of labelling agents is strictly optional. It is further expected that the methods and improved DEP devices of the disclosure can be further used to uncover characteristics and mechanisms associated to drug-resistance. Accordingly, the methods and improved DEP devices of the disclosure can be used for applications, such as drug discovery and drug screening applications.

In a particular embodiment, the disclosure provides a dielectrophoresis (DEP) device capable of high-throughput continuous dielectrophoretic cell separation or sorting comprising: one or more inlet channels that can accommodate a fluid input comprising cells; optionally, one or more filters that are in fluid communication with the one or more inlet channels and one or more hydrophoretic modules, wherein the one or more filters are configured to prevent passage of cell aggregates from the fluid input; optionally, a cell mixing section in fluid communication with the one or more inlet channels and one or more hydrophoretic modules, wherein the cell mixing section distributes the cells more evenly in the fluid input before flowing into the hydrophoretic module; one or more hydrophoretic modules that are in fluid communication with the one or more inlet channels and one or more dielectrophoretic modules, wherein the hydrophoretic modules comprise a serpentine channel structure, and wherein the hydrophoretic modules are configured to focus cells into two streams along the edges of the serpentine channel structure; one or more dielectrophoretic modules comprising an electrode array that are in fluid communication with the hydrophoretic modules and the outlets, wherein the dielectrophoretic modules separate cells by their inherent cell electrophysiological properties, and wherein the dielectrophoretic modules comprise one or more of structural features (i), (ii), (iii) and/or (iv): (i) the electrode array comprises 2 or more electrodes; (ii) the electrodes having a width from 25 μm to 500 μm; (iii) the electrodes having a tip radius of greater than 50 μm; and/or (iv) the gap between the electrodes in the electrode array is nonuniform in size; a plurality of outer outlets in fluid communication with the one or more dielectrophoretic modules that are configured to collect cells that were not focused by DEP; and one or more inner outlets in fluid communication with the one or more dielectrophoretic module that are configured to collect cells that were focused by DEP; wherein the plurality of outer outlets and the one or more inner outlets have a diameter that exceeds 1500 μm; and wherein the focused cells of the one or more inner outlets have different dielectric properties than the unfocused cells in the plurality of outer outlets. In another embodiment, the DEP device is made from two substrate layers that comprise formable materials that are aligned and connected or bonded together. In yet another embodiment, the formable materials are selected from gold, chromium, titanium, indium tin oxide (ITO), glass, poly dimethylsiloxane (PDMS), polystyrene (PS), polyether ether ketone (PEEK), polyethylene terephthalate (PET), polyvinyl chloride (PVC), polymethylmethacrylate (PMMA), cyclic olefin copolymer (COC), polycarbonate (PC), and polyetherimide (PEI). In a further embodiment, at least one of the substrates comprises hydrophoretic features with multiple independent heights, wherein the dielectrophoretic module has a microfluidic channel height that is modified to be less than the overall height of the hydrophoretic features. In yet a further embodiment, the DEP device further comprises a cell delivery chamber that allows intermittent or continuous mixing of solutions and is reversibly attachable to the one or more inlet channels, wherein the cell delivery channel is a pressurized chamber that is reversibly attachable to a pressure exerting device. In another embodiment, the DEP device filters comprise one or more filters, and wherein the one or more filters are an array of raised structures that have defined gap sizes between the raised structures. In yet another embodiment, the DEP device comprises the cell mixing section located before the hydrophoretic module, and wherein the cell mixing section mixes by using hydrophoretic mixing, or acoustic actuated mixing. In a further embodiment, the walls of the serpentine channel structure of the hydrophoretic modules are greater than 10 μm in width. In another embodiment, the serpentine channel structure of the hydrophoretic modules comprises microstructures that changes the cross-sectional area of the channel structure to align the cells into two streams along the channel edges. In yet another embodiment, for structural feature (ii), the width of the electrodes is from 50 μm to 400 μm. In a further embodiment, for structural feature (iii), the electrode tip radius is from 100 μm to 250 μm. In yet a further embodiment, for structural feature (iv), the gap between the electrodes is variable along the lengths of the electrodes, wherein the gap is narrowest at the base of the electrodes, and most wide at the tip of the electrodes. In another embodiment, the DEP device comprises one inlet channel, at least 2 hydrophoretic modules; at least 2 dielectrophoretic modules; at least 2 inner outlets; and at least 4 outer outlets.

In a particular embodiment, the disclosure also provides a method to sort or separate a heterogenous population of cells into two separate populations of cells based upon differences in their dielectric properties, the method comprising: providing a DEP buffer comprising a heterogeneous population of cells into the one or more inlet channels of a DEP device disclosed herein; dissociating the heterogeneous population of cancer cells into single cells in the hydrophoretic modules; and separating the single cells using the one or more dielectrophoretic modules into a focused cell population in the one or more inner output channels and non-focused cell population in the plurality of the outer output channels. In yet another embodiment, wherein the focused cell population has higher Cspec values than the non-focused cell population. In a certain embodiment, the DEP buffer comprises a ROCK-pathway inhibitor. In a further embodiment, the ROCK-pathway inhibitor is Y-27632 or Chroman 1. In yet a further embodiment, the heterogeneous population of cells comprise cancer cells. In another embodiment, the cancer cells are derived from a cancer selected from glioblastoma, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, including triple negative breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, Merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, soft tissue sarcoma, uterine cancer, uterine sarcoma, skin cancer (non-melanoma), skin cancer (melanoma), papillomas, actinic keratosis and keratoacanthomas, merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor. In yet another embodiment, the cancer cells comprise cancer cells that have drug resistance to an anticancer agent and cancer cells that do not have drug resistance to the anticancer agent. In a further embodiment, the anticancer agent is selected from angiogenesis inhibitors, tyrosine kinase inhibitors, PARP inhibitors, alkylating agents, vinca alkaloids, anthracyclines, antitumor antibiotics, antimetabolites, topoisomerase inhibitors, aromatase inhibitors, mTor inhibitors, retinoids, and HDAC inhibitors.

In a particular embodiment, the disclosure provides a dielectrophoresis (DEP) device capable of high-throughput continuous dielectrophoretic cell separation or sorting comprising: one or more inlet channels that can accommodate a fluid input comprising cells; optionally, one or more filters that are in fluid communication with the one or more inlet channels and one or more hydrophoretic modules, wherein the one or more filters are configured to prevent passage of cell aggregates from the fluid input; optionally, a cell mixing section in fluid communication with the one or more inlet channels and one or more hydrophoretic modules, wherein the cell mixing section distributes the cells more evenly in the fluid input before flowing into the hydrophoretic module; one or more hydrophoretic modules that are in fluid communication with the one or more inlet channels and one or more dielectrophoretic modules, wherein the hydrophoretic modules comprise a serpentine channel structure, and wherein the hydrophoretic modules are configured to focus cells into two streams along the edges of the serpentine channel structure; one or more dielectrophoretic modules comprising an electrode array that are in fluid communication with the hydrophoretic modules and the outlets, wherein the dielectrophoretic modules separate cells by their inherent cell electrophysiological properties, and wherein the dielectrophoretic modules comprise one or more of structural features (i), (ii), (iii) and/or (iv): (i) the electrode array comprises 2 or more electrodes; (ii) the electrodes having a width from 25 μm to 500 μm; (iii) the electrodes having a tip radius of greater than 50 μm; and/or (iv) the gap between the electrodes in the electrode array is nonuniform in size; a plurality of outer outlets in fluid communication with the one or more dielectrophoretic modules that are configured to collect cells that were not focused by DEP; and one or more inner outlets in fluid communication with the one or more dielectrophoretic module that are configured to collect cells that were focused by DEP; wherein the plurality of outer outlets and the one or more inner outlets have a diameter that exceeds 1500 μm; and wherein the focused cells of the one or more inner outlets have different dielectric properties than the unfocused cells in the plurality of outer outlets. In a further embodiment, the DEP device is made from two substrate layers that are aligned and connected or bonded together, particularly, wherein the two substrate layers are irreversibly bonded together. In yet a further embodiment, the two substrate layers comprises formable materials that are aligned with or without alignment marks and are connected or bonded together, particularly, wherein the formable materials are selected from chromium, titanium, indium tin oxide (ITO), glass, polydimethylsiloxane (PDMS), polystyrene (PS), polyether ether ketone (PEEK), polyethylene terephthalate (PET), polyvinyl chloride (PVC), polymethylmethacrylate (PMMA), cyclic olefin copolymer (COC), polycarbonate (PC), and polyetherimide (PEI). In another embodiment, the formable materials are thermoplastic materials or thermosetting materials, particularly, wherein the thermoplastic material or the thermosetting material is selected from poly dimethylsiloxane (PDMS), polystyrene (PS), polyether ether ketone (PEEK), polyethylene terephthalate (PET), polyvinyl chloride (PVC), polymethylmethacrylate (PMMA), cyclic olefin copolymer (COC), polycarbonate (PC), and polyetherimide (PEI), more particularly, wherein the thermosetting material is polydimethylsiloxane (PDMS). In yet another embodiment, at least one of the substrate layers comprises alignment marks to facilitate proper alignment when the two substrates are connected or bonded together, particularly, wherein at least one of the substrates layers comprises two sets of alignment marks, a first set comprising macro-alignment marks which allows for quick orientation of the two substrate layers, and a second set comprising microalignment marks which allows for fine tuning aligning of the two substrates layers. IN a further embodiment, the DEP device further comprises a cell delivery chamber that allows intermittent or continuous mixing of solutions is reversibly attachable to the one or more inlet channels, wherein the cell delivery channel is a pressurized chamber that is reversibly attachable to a pressure exerting device, particularly, wherein the pressure exerting device is a pump, more particularly, wherein the pressure exerting device is a fluidic pump. In yet a further embodiment, the DEP device filters comprise one or more filters, and wherein the one or more filters are an array of raised structures that have defined gap sizes between the raised structures, particularly, wherein the raised structures are pillars or columns, more particularly, wherein the one or more filters comprises 2 or 3 series of pillars or columns that have different sized gaps between the pillar or columns, more particularly, wherein the one or more filters comprises 2 or 3 series of pillars or columns wherein the series of pillars columns nearest the hydrophoretic module has the smallest gaps between the pillars or columns, and the series of pillars or columns furthest from the hydrophoretic module has the largest gaps between the pillars or columns. In another embodiment, the DEP device comprises the cell mixing section located between the filter and the hydrophoretic module, and wherein the cell mixing section mixes by using hydrophoretic mixing, or acoustic actuated mixing, particularly, wherein the cell mixing section mixes by using hydrophoretic mixing. In yet another embodiment, the walls of the serpentine channel structure of the hydrophoretic modules have a width that is greater than 10 μm, particularly, wherein the hydrophoretic modules comprises gaps that are greater than 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 350 μm, 400 μm, 450 μm, 500 μm, 550 μm, 600 μm, 650 μm, 700 μm, 750 μm, 800 μm, 850 μm, 900 μm, 950 μm, or 1000 μm, or a range that includes or is between any two of the foregoing values. In a further embodiment, the serpentine channel structure of the hydrophoretic modules comprises microstructures that changes the cross-sectional area of the channel structure to align the cells into two streams along the channel edges, particularly, wherein the microstructures are from 30 μm to 70 μm in height. In yet a further embodiment, at least one of the substrates comprises hydrophoretic features with multiple independent heights, wherein the dielectrophoretic module has a microfluidic channel height that is modified to be less than the overall height of the hydrophoretic features. In another embodiment, the dielectrophoretic module comprises structural features (i), (ii), (iii) and (iv). In yet another embodiment, the DEP device comprises the cell mixing section located between the filter and the hydrophoretic module, and wherein the cell mixing section mixes by using hydrophoretic mixing, or acoustic actuated mixing, particularly, wherein the cell mixing section mixes by using hydrophoretic mixing. In a further embodiment, the walls of the serpentine channel structure of the hydrophoretic modules comprises a width greater than 10 μm, particularly, wherein the walls of the serpentine channel structure of the hydrophoretic modules comprises a width that is greater than 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 350 μm, 400 μm, 450 μm, 500 μm, 550 μm, 600 μm, 650 μm, 700 μm, 750 μm, 800 μm, 850 μm, 900 μm, 950 μm, or 1000 μm, or a range that includes or is between any two of the foregoing values. In yet a further embodiment, the serpentine channel structure of the hydrophoretic modules comprises ridges and trenches to generate a diverging fluid flow that focuses cells into two streams along the channel edges, particularly, wherein the ridges are from 30 μm to 70 μm in height. In another embodiment, the dielectrophoretic module has a microfluidic channel height that is modified to be the height of only one of the substrate layers. In yet another embodiment, the dielectrophoretic module comprises structural features (i), (ii), (iii) and (iv). In a further embodiment, for structural feature (i), the array of electrodes comprises at least 4, 8, 12, 16, 20, 24, 28, 30, 34, 38, 40, 44, 48, 50, 54, 58, 60, 64, 68, 70, 74, 78, 80, 90, 100, 110, 120, 130, 140, 150, 160, 170, 180, 200, 210, 220, 230, 240, 250, 260, 270, 280, 300, 310, 320, 330, 340, 350, 360, 370, 380, 400, 410, 420, 430, 440, 450, 460, 470, 480, or 500 electrodes, or a range of electrodes that includes or is between any two of the foregoing values, particularly, wherein for structural feature (i), the array of electrodes comprises more than 40 electrodes. In yet a further embodiment, for structural feature (ii), the width of the electrodes is selected from 50 μm, 55 μm, 60 μm, 65 μm, 70 μm, 75 μm, 80 μm, 85 μm, 90 μm, 95 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 350 μm, 400 μm, and 500 μm, or a range of widths that includes or is between any two of the foregoing values, particularly, wherein for structural feature (i), the array of electrodes comprises from 50 μm to 400 μm. In a certain embodiment, for structural feature (iii), the electrode tip radius is 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, or 300 μm, or a range of radii that includes or is between any two of the foregoing values, particularly, wherein for structural feature (ii), the electrode tip radius is from 100 μm to 250 μm. In a further embodiment, for structural feature (iv), the gap between the electrodes is variable along the lengths of the electrodes, wherein the gap is narrowest at the base of the electrodes, and most wide at the tip of the electrodes. In yet a further embodiment, the DEP device comprises 2 to 4 of outer outlets that are radially orientated from the end of the dielectrophoretic module, particularly, wherein the DEP device comprises 2 or 4 of outer outlets. In another embodiment, the diameter of the plurality of outer outlets and the one or more inner outlets are greater than 1500 μm, 1510 μm, 1520 μm, 1530 μm, 1540 μm, 1550 μm, 1560 μm, 1570 μm, 1580 μm, 1590 μm, 1600 μm, 1610 μm, 1620 μm, 1630 μm, 1640 μm, 1650 μm, 1660 μm, 1670 μm, 1680 μm, 1690 μm, 1700 μm, 1710 μm, 1720 μm, 1730 μm, 1740 μm, 1750 μm, 1760 μm, 1770 μm, 1780 μm, 1790 μm, 1800 μm, 1850 μm, 1900 μm, 1950 μm, 2000 μm, 2500 μm, 3000 μm, 3500 μm, 4000 μm, 4500 μm, or 5000 μm, or a range that includes or is between any two of the foregoing diameters, particularly wherein the diameter of the plurality of outer outlets and the one or more inner outlets are greater than 2000 μm. In yet another embodiment, the focused cells of inner outlet have different specific membrane capacitance (Cspec) values than the unfocused cells in the plurality of outer outlets, particularly, wherein the focused cells of inner outlet have higher Cspec values than the unfocused cells in the plurality of outer outlets. In a further embodiment, the DEP device comprises one inlet channel, at least 2 hydrophoretic modules; at least 2 dielectrophoretic modules; at least 2 inner outlets; and at least 4 outer outlets. In yet a further embodiment, the DEP device comprises one inlet channel; 4 hydrophoretic modules; 4 dielectrophoretic modules; 4 inner outlets; and at least 8 outer outlets.

In a particular embodiment, the disclosure also provides a method to sort or separate a heterogenous population of cells into two separate populations of cells based upon differences in their dielectric properties, the method comprising: providing a DEP buffer comprising a heterogeneous population of cells into the one or more inlet channels of a DEP device disclosed herein; dissociating the heterogeneous population of cancer cells into single cells in the hydrophoretic modules; separating the single cells using the one or more dielectrophoretic modules into a focused cell population in the one or more inner output channels and non-focused cell population in the plurality of the outer output channels, particularly, wherein the dielectrophoretic modules use alternating and/or direct current. In another embodiment, the DEP buffer comprises a ROCK inhibitor. In yet another embodiment, the ROCK inhibitor is Y-27632 or Chroman 1. In a further embodiment, the population of heterogeneous cells comprise cancer cells. In yet a further embodiment, the cancer cells are derived from a cancer selected from adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, including triple negative breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, soft tissue sarcoma, uterine cancer, uterine sarcoma, skin cancer (non-melanoma), skin cancer (melanoma), papillomas, actinic keratosis and keratoacanthomas, merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor. In another embodiment, the cancer cells comprise cancer cells that have drug resistance and cancer cells that do not have drug resistance. In yet another embodiment, the drug resistance is resistance to an anticancer agent. In a further embodiment, the anticancer agent is selected from angiogenesis inhibitors, tyrosine kinase inhibitors, PARP inhibitors, alkylating agents, vinca alkaloids, anthracyclines, antitumor antibiotics, antimetabolites, topoisomerase inhibitors, aromatase inhibitors, mTor inhibitors, retinoids, and HDAC inhibitors. In a certain embodiment, the cancer cells are glioblastoma cancer cells. In a further embodiment, a portion of the glioblastoma cells have drug resistance, and a portion of the glioblastoma cells do not have drug resistance. In yet a further embodiment, the portion of glioblastoma cells that have drug resistance are resistant to a drug selected from temozolomide, bevacizumab, altiratinib, panobinostat, trebanaib, enzastaurin, crenolanib, tandutinib, mibefadil, gliadel, and afatinib. In a certain embodiment, the portion of glioblastoma cells have drug resistance to temozolomide.

In a particular embodiment, the disclosure provides for a dielectrophoresis (DEP) device capable of high-throughput continuous dielectrophoretic separation comprising: an inlet channel that can be loaded with a DEP buffer comprising a population of cells; a filter to remove cell clumps in fluid communication with the inlet channel; a hydrophoretic module that is in fluid communication with the filter, where the hydrophoretic module comprises a serpentine structure, wherein the gaps in the serpentine structure are enlarged to improve the bonding surface; a dielectrophoretic module comprising an electrode array that is in fluid communication with the hydrophoretic alignment; one or more outer channels in fluid communication with the dielectrophoretic module; and an inner channel in fluid communication with the electrode array; wherein when cells are inputted into the inlet channel, the cells sorted in the outer channels are unfocused cells while the cells sorted in the inner channel are focused cells; and wherein the focused cells of inner channel have different specific membrane capacitance (Cspec) values than the unfocused cells in the outer channels. In a further embodiment, the DEP device has one or more of the following or additional structural design features of (1) to (10): (1) the DEP device comprises macro alignment marks to improve assembly speed; (2) the DEP device comprises micro alignment marks to improve assembly speed and assignment precision; (3) the DEP device comprises expanded outlet diameters to reduce sample collection frequency; (4) the DEP device comprises a modified electrode tip radius to improve cell release at the electrode tip; (5) the DEP device comprises more outlets while maintaining equal fluid pressure among the outlet channels, which enable finer separation into multiple sorted cell fractions; (6) the DEP device further comprises a cell mixing section before the hydrophoretic module to prolong the consistency of the hydrophoretic module; (7) the DEP device comprises a greater series of electrodes in the electrode array configured to increase throughput, reduce the operating voltage, and improve sensitivity; (8) the DEP device comprises a series of electrodes in the electrode array configured have varying electrode gaps to improve separation resolution; (9) the DEP device comprises a series of electrodes in the electrode array that are configured to have increased widths in the electrode array so as to improve DEP focusing force while maintaining cell viability; and/or (10) the channel heights of the DEP device are configured so as to reduce the operating voltage to maintain cell viability and promote uniform separation. In yet a further embodiment, the DEP device has the structural design features of (1) to (10). In another embodiment, the population of cells comprise cancer cells. In yet another embodiment, the cancer cells are derived from a cancer selected from adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, including triple negative breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, soft tissue sarcoma, uterine cancer, uterine sarcoma, skin cancer (non-melanoma), skin cancer (melanoma), papillomas, actinic keratosis and keratoacanthomas, merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor. In certain embodiment, the cancer is glioblastoma. In a further embodiment, the cancer cells comprise cancer cells that are resistant to a drug. In yet a further embodiment, the DEP device can sort the cancer cells into populations of drug resistant cancer cells and non-drug resistant cancer cells. In another embodiment, the drug is an anticancer agent. Examples of anticancer agents, include, but are not limited to, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and tiimethylolomelamine; acetogenins (e.g., bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBT-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; vinca alkaloids; epipodophyllotoxins; antibiotics such as the enediyne antibiotics (e.g., calicheamicin, especially calicheamicin gammall and calicheamicin omegall; L-asparaginase; anthracenedione substituted urea; methyl hydrazine derivatives; dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitiaerine; pentostatin; phenamet; pirarubicin; losoxantione; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2 2″-trichlorotiiethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® (docetaxel) (Rhone-Poulenc Rorer, Antony, France); chloranbucil; GEMZAR® (gemcitabine); 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (e.g., CPT-11); topoisomerase inhibitor RFS 2000; difluoromethylomithine (DFMO); retinoids such as retinoic acid; capecitabine; leucovorin (LV); irenotecan; adrenocortical suppressant; adrenocorticosteroids; progestins; estrogens; androgens; gonadotropin-releasing hormone analogs; and pharmaceutically acceptable salts, acids or derivatives of any of the above. Also included anticancer agents are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON-toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASL® exemestane, formestanie, fadrozole, RIVISOR® vorozole, FEMARA® letrozole, and ARTMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF-A expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rJL-2; LURTOTECAN® topoisomerase 1 inhibitor; ABARELLX® rmRH; antibodies such as trastuzumab and pharmaceutically acceptable salts, acids or derivatives of any of the above. In a further embodiment, the anticancer agent is selected from angiogenesis inhibitors, tyrosine kinase inhibitors, PARP inhibitors, alkylating agents, vinca alkaloids, anthracyclines, antitumor antibiotics, antimetabolites, topoisomerase inhibitors, aromatase inhibitors, mTor inhibitors, retinoids, and HDAC inhibitors. In a certain embodiment, the anticancer agent is temozolomide.

In a particular embodiment, the disclosure also provides a method to sort drug-resistant cancer cells from non-drug-resistant cancer cells comprising: inputting a DEP buffer comprising a population of cancer cells into the DEP device of any one of the preceding claims, wherein the population of cancer cells comprise a portion of cells that are drug-resistant and a portion of cells that are not drug-resistant; dissociating the population of cancer cells into single cancer cells in the DEP device; separating the cells into a focused cancer cell population in the inner channel and non-focused cancer cell populations in the outer channel using the DEP device; measuring the Cspec values on the focused cancer cell populations and non-focused cancer cell populations to confirm sorting was successful; and performing an assay to assess the drug resistance of the sorted cancer cell populations. In a further embodiment, the DEP buffer comprises a ROCK inhibitor. In yet a further embodiment, the ROCK inhibitor is Y-27632 or Chroman 1. In another embodiment, the cancer cell population is a population of glioblastoma cancer cells. In yet another embodiment, the drug resistance is to the drug temozolomide.

The details of one or more embodiments of the disclosure are set forth in the accompanying drawings and the description below. Other features, objects, and advantages will be apparent from the description and drawings, and from the claims.

### DESCRIPTION OF DRAWINGS

FIG. 1A-E demonstrates that incubation of D54 cells in DEP buffer can decrease viability. (A) Schematic describes experimental paradigm to test D54 viability after incubation in cell culture media or DEP buffer. Adherent D54 cells were dissociated then resuspended in media or DEP buffer and incubated on ice or at room temperature (RT). Cell viability was tested immediately after incubation by trypan blue. Equivalent numbers of cells were plated per condition and adherent cells observed after 1-2 days by phase contrast microscopy to assess cell recovery. (B) Cell viability after 6-hour incubation remained high for all conditions. Viability of D54 cells in DEP buffer may be slightly lower than that of cells in media. N=1. (C) Phase-contrast images of D54 cells stained with trypan blue after 6-hour incubation show high cell viability and increased clustering of cells incubated in media at RT. (D) Phase contrast images of adherent D54 cells one day after 6-hour incubation in media or DEP buffer. Cells incubated in media at RT show the best recovery, indicated by cell number and cell morphology. (E) Phase contrast images of adherent D54 cells one day after 0-6 hour incubation in media or DEP buffer. Cell recovery decreases with increasing incubation time in DEP buffer.

FIG. 2A-D presents optimization of buffer conditions for DEP-based sorting of GBM cells. (A) Viability of D54 cells immediately after 6-hour RT incubation in regular growth media, DEP buffer, or DEP buffer supplemented with 5 μM ROCKi was measured by trypan blue staining. Addition of ROCKi improved viability in the DEP buffer. (B) After 6-hour incubation in media or buffers, D54 cells were plated in normal growth conditions and the number of cells measured after 2 days by XTT assay. DEP buffer with 5 μM ROCKi significantly increased the number of viable cells compared to DEP buffer alone. (C) Incubation of D54 cells for 6 hours in DEP buffer or DEP buffer with 5 μM ROCKi does not change membrane capacitance or cell diameter measured after incubation. Acutely isolated cells (Media control, 0 h) served as a control. (D) D54 cells were plated after 6-hour incubation in buffers and after 1 day were exposed to TMZ to assess TMZ resistance. TMZ IC₅₀ is shown as fold change relative to media control, 0 h, and no difference in TMZ resistance was detected across samples. Error bars show SD. N≥3, one-way ANOVA, Tukey post hoc for multiple comparisons, *p<0.05, **p<0.01, ***p<0.001, ****p<0.0001.

FIG. 3A-D demonstrates optimization of ROCKi concentration in DEP buffer to support viability of GBM cells. (A) Viability of D54 cells immediately after 6-hour incubation in regular growth media, DEP buffer, or DEP buffer supplemented with 1, 5, or 10 μM ROCKi was measured by trypan blue staining. Acutely isolated cells (Media control, 0 h) served as a control. Addition of 1 or 5 μM ROCKi improved viability in the DEP buffer. (B) Incubation of D54 cells for 6 hours in DEP buffer with 1 μM ROCKi decreased membrane capacitance compared to control cells in media for 0 hours, but there was no change in the capacitance of cells in the other conditions. (C) There was no significant difference in the diameters of D54 cells in any of the conditions. (D) There was no significant difference in TMZ resistance as measured by fold change in IC₅₀ relative to acutely isolated cells (Media control, 0 h). Error bars show SD. N≥3, one-way ANOVA, Dunnett's post hoc for multiple comparisons to media control (0 h A, B, C; and 6 h D), *p<0.05.

FIG. 4A-B demonstrates the effects of alternative DEP buffers on glioma cell viability. DEP buffer (DB): 8.5% w/v sucrose, 0.3% w/v glucose, 0.725% RPMI. RBC DEP Buffer (RBC-DB): 250 mM sucrose, 17 mM glucose, 0.1 mM CaCl₂). DB and RBC-DB buffers were adjusted to 100 S/cm. (A) Cells were incubated in media or DEP buffers for 6 hours. Acutely isolated cells (Media control, 0 h) served as a control. After incubation, cell viability was checked by trypan blue staining. Cell viability was high for every DEP buffer tested. (B) Cells were plated after incubation and the confluency (% culture surface area covered by cells) of adherent cells measured 1 day later using ImageJ. Among cells incubated in DEP buffers, those in DEP buffer supplemented with 5 μM ROCKi demonstrated the most confluency. N=1.

FIG. 5A-B demonstrates that while the control and TMZ resistant (TR) cell lines differ in size, the isolated TMZ resistant cells were of similar size. (A) Analysis of phase contrast images of cells and forward scatter profiles in flow cytometry show that TR cells are significantly larger than controls. (B) Image analysis of D54 cells sorted by DEP show that unsorted controls, focused and unfocused cells do not differ in size. Error bars show SD. N≥3, one-way ANOVA, Tukey post hoc for multiple comparisons, *p<0.05, ****p<0.0001.

FIG. 6A-C demonstrates TMZ resistant cells and controls differ in membrane electrophysiological properties. (A) DEP spectra of D54 (D54, D54-DMSO, D54-TR) and U251 (U251, U251-DMSO, U251-TR) cells show the relative DEP force across a range of applied frequencies. For both sets of cells, DEP spectra of TMZ resistant (TR) cells are right shifted compared to those of controls. (B) The specific membrane capacitance (Cspec) values for TR cells are lower than those of controls. (C) The midpoint membrane frequency of TR cells is higher than those of controls. Error bars show SEM. N≥3, one-way ANOVA, Tukey post hoc for multiple comparisons, **p<0.01, ***p<0.001, ****p<0.0001.

FIG. 7A-C presents an exemplary DEP device of the disclosure. (A) Schematics of a novel DEP device are labeled with letters to denote the expanded views (B, C) and numbers to show the main improved features. Relevant dimensions are included in the table. (B) 3D Cross-section view of hydrophoretic alignment section with a fluid element to show new high configuration of microfluidic channel. (C) Schematics of different electrode configurations show an example of expanding electrode gap geometry and enlarged electrode width. All schematics are not drawn to scale.

FIG. 8 provides an embodiment of a DEP device of the disclosure. Schematics of the DEP device are labeled with numbers to show key features listed in the table. The schematic is not drawn to scale.

FIG. 9 presents a pressurized cell delivery chamber for fluidic pumps. The schematics illustrates the working mechanism of the pressurized cell delivery chamber. A fluidic pump pressurizes the sealed chamber by pushing fluid into it from the top. The increased pressure in the chamber causes the suspended cells to leave the chamber from the bottom. The crossed arrows indicate intermittent mixing to maintain cells in suspension.

FIG. 10 presents an embodiment of a DEP device of the disclosure. The DEP device as shown has an increased serpentine gap to improve bonding surface; and has an increased outlet diameter to improve collection volume.

FIG. 11 presents an embodiment of a DEP device of the disclosure. The DEP device as shown has an increased serpentine gap to improve bonding surface; has an increased outlet diameter to improve collection volume; has double electrode number (from 20 pairs to 40 pairs); and has increased electrode tip radius to improve cell release at the electrode tip.

FIG. 12 presents an embodiment of a DEP device of the disclosure. The DEP device as shown has an increased serpentine gap to improve bonding surface; has an increased outlet diameter to improve collection volume; has a parallel design to increase throughput; and has a 3D electrode connection for ease of fabrication.

FIG. 13 presents an embodiment of a DEP device of the disclosure. The DEP device as shown has an increased serpentine gap to improve bonding surface; has an increased outlet diameter to improve collection volume; has double electrode number (from 20 pairs to 40 pairs); has an increased electrode tip radius to improve cell release at the electrode tip; has an alternative inlet layout to improve cell entry; has a parallel design to increase throughput; and has a 3D electrode connection for ease of fabrication.

FIG. 14 presents an embodiment of a DEP device of the disclosure. The DEP device as shown has an increased serpentine gap to improve bonding surface; has an increased outlet diameter to improve collection volume; has an increased electrode tip radius to improve cell release at the electrode tip and has an increased electrode width to 300 um to improve DEP focusing force.

FIG. 15 presents an embodiment of a DEP device of the disclosure. The DEP device as shown has an increased serpentine gap to improve bonding surface; an increased outlet diameter to improve collection volume; has double electrode number (from 20 pairs to 40 pairs); has an increased electrode tip radius to improve cell release at the electrode tip; and has an increased outlet number to improve separation purity.

FIG. 16 presents an embodiment of a DEP device of the disclosure. The DEP device as shown has an increased serpentine gap to improve bonding surface; has an increased outlet diameter to improve collection volume; has an increased electrode tip radius to improve cell release at the electrode tip; and has an added an extra inlets to help remove debris.

FIG. 17 presents an embodiment of a DEP device of the disclosure. The DEP device as shown has an increased serpentine gap to improve bonding surface; has an increased outlet diameter to improve collection volume; has double electrode number (from 20 pairs to 40 pairs); has an increased electrode tip radius to improve cell release at the electrode tip; and includes a pre-hydrophoretic focusing section to redirect cells to the center to improve hydrophoretic focusing consistency.

FIG. 18 presents an embodiment of a DEP device of the disclosure. The DEP device as shown has an increased serpentine gap to improve bonding surface; has an increased outlet diameter to improve collection volume; has double electrode number (from 20 pairs to 40 pairs); has an increased electrode tip radius to improve cell release at the electrode tip; includes a pre-hydrophoretic focusing section to redirect cells to the center to improve hydrophoretic focusing consistency; and an increased outlet number to improve separation purity.

FIG. 19 presents an embodiment of a DEP device of the disclosure. The DEP device as shown is a dielectrophoresis rounded electrode array microfluidic sorter (DREAMS) device. The gradual increasing electrode gap enables continuous cell release based on cell properties, which improves separation resolution.

FIG. 20A-G provides for isolation of TMZ resistant cells by a DEP device of the disclosure. (A) GBM cells were sorted into focused and unfocused fractions in the DEP device. Cells in the unfocused fraction had lower membrane capacitance values than cells in the focused fraction or controls in DEP buffer. (B, D) D54 and U251 GBM cells sorted in the DEP device show lower membrane capacitance values for the unfocused cells compared to cells in the focused fraction. (C, E) D54 and U251 Cells in the unfocused fraction are more resistant to TMZ than those in the focused fraction. (F) DB70 patient derived GBM cells sorted in the DEP device show lower membrane capacitance values for the unfocused cells compared to cells in the focused fraction. (G) DB70 cells in the focused fraction are more resistant to TMZ than those in the unfocused fraction. Error bars show SD. N≥3 (except D, E), all analyses one-way ANOVA (except G, paired t test), Tukey post hoc for multiple comparisons, *p<0.05, **p<0.01, ***p<0.001.

FIG. 21 evaluates membrane capacitance of GBM cell lines (D54 and 251) using a DEP device of the disclosure. Using the DEP device, the dielectric properties of two glioma cell lines were compared. TMZ resistant cells that were derived from those parent cells, which are labeled with TR, and their DMSO control. When compared the specific membrane capacitance of these cells, it was found that both TR cell lines are lower in capacitance in comparison to their parent cells. Additionally, it was found the midpoint membrane frequency of the TR cells are significantly greater than that of the parent cells. These results indicate Cspec and MMF can be used as biophysical markers to identify TMZ resistance cells using DEP.

FIG. 22 evaluates cell diameters of TMZ-resistant and non-resistant GBM cell lines (D54 and 251). When compared the size of these cell populations and found similar size distribution which indicate separation based on size is not ideal. On the other hand, it was found some overlap in Cspec between both parent and TMZ resistant cell lines. Therefore, it was postulated DEP could enrich TMZ tolerant cells.

FIG. 23 provides a general workflow for using a DEP device of the disclosure to isolate TMZ-resistant cancer cells. First, the population of cells are dissociated into single cells. Then, the single cells are separated into a focused and unfocused population using the DEP device. The Cspec is measured to confirm sorting was successful, and an XTT assay is performed to assess the TMZ resistance of the sorted populations.

FIG. 24 demonstrates that patient-derived GBM cells (DB70) sorted for TMZ-resistant cells, maintained enrichment over passaging post sorting.

### DETAILED DESCRIPTION

As used herein and in the appended claims, the singular forms “a,” “an,” and “the” include plural referents unless the context clearly dictates otherwise. Thus, for example, reference to “a cell” includes a plurality of such cells and reference to “the cell sorter” includes reference to one or more cell sorters and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

For purposes of the disclosure the term “cancer” will be used to encompass cell proliferative disorders, neoplasms, precancerous cell disorders and cancers, unless specifically delineated otherwise. Thus, a “cancer” refers to any cell that undergoes aberrant cell proliferation that can lead to metastasis or tumor growth. Exemplary cancers include but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodermal tumors, visual pathway and hypothalamic glioma, breast cancer, including triple negative breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, soft tissue sarcoma, uterine cancer, uterine sarcoma, skin cancer (non-melanoma), skin cancer (melanoma), papillomas, actinic keratosis and keratoacanthomas, merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

Glioblastoma (GBM) is an aggressive type of cancer that can occur in the brain or spinal cord. Resistance of GBM to the first-line chemotherapy agent temozolomide (TMZ) is a major problem. The rapid identification and enrichment of TMZ-resistant cells from resected patient tumors could lead to a better understanding of their molecular characteristics, potential mechanisms of resistance, and the development of more effective treatment. Conventional cell sorting systems, such as fluorescent-activated cell sorting (FACS) and magnetic-activated cell sorting (MACS), are label-based sorting methods that rely on well-defined biomarkers that are often not available for many important cell types, including drug-resistant GBM cells. One approach for enriching TMZ-resistant cells for analysis involves contacting tumor cells with increasing concentrations of TMZ to select for resistant cells. The selection approach can take from weeks to months; and cellular changes could occur due to the prolonged TMZ exposure that do not directly correlate with resistance or do not reflect the phenotype of resistant cells in the native tumor environment. Furthermore, patient-derived cells are notorious for being difficult to culture in vitro after resection. A means to rapidly enrich TMZ-resistant cells for analysis would bypass extended culture in TMZ and avoid many of these issues.

A widely accepted theory for the occurrence of drug-resistance is through the process of selection. Within the tumor niche there is a heterogeneous population of cells that have varying degrees of tolerance to TMZ that allows a portion of cells to survive the initial treatment, and which, over time acquire additional mutations that provide resistance to the drug. By being able to rapidly sort out pre-existing tolerant cells would enable the characterization of this cell population so as to elucidate the cells' properties and study the mechanisms of resistance, so as to develop more effective treatments for patients. However, since little is known about these pre-existing tolerant cells, there is a lack of reliable biomarkers to enrich these cells through traditional biomarker-based cell sorting techniques, such as FACS or MACS.

The subtle phenotypic differences between cells can be difficult to detect but have big consequences for cell behavior. Separating cells based on their phenotypic differences enables critical experiments aimed at deciphering their biological functions and determining their relevance in disease. Cell separation systems that do not require cell-type-specific labels have a number of advantages. Labels can be limiting since many cells of interest for biological or biomedical applications do not have sufficient markers that distinguish them from other cell types. Labeling of cells could change their biological function, and since this is rarely screened for or tested, incorrect assumptions may be made about the function of labeled cells. Antibodies or labels used for traditional flow cytometry methods bind to cell surface components and could stimulate intracellular signaling cascades. Labeling of intracellular components requires modification of the cell to introduce foreign material that may interfere with normal cellular function. Unlabeled and unmodified cells are also ideal for therapeutic purposes since they require less manipulation that could affect cell phenotype prior to introduction into a patient. Continued development of label-free cell separation technologies will provide much needed alternatives to label-based separation systems

Many different microfluidic cell separation devices have been developed. Combining multiple separation modalities in microfluidic devices can have advantages over any single approach. Label-free systems include hydrophoresis, in which fluid flow is used to direct cell location in a microfluidic channel, and dielectrophoresis (DEP), in which nonuniform electric fields induce cell movement due to inherent cellular properties. Hydrophoresis may not have sufficient resolving power to separate cells that are quite similar to each other, particularly cells that are of similar size. DEP can distinguish cells of similar size as long as the cells have distinct electrophysiological properties. For example, similarly sized cells that significantly differ in membrane capacitance can be separated by alternating current (AC) DEP in the frequency range of approximately 1-1000 kHz. A limitation to DEP-based sorting is that many DEP devices rely on trapping of cells along electrode arrays and release of the isolated cells after washing away nontrapped cells. This “trap and release” mechanism has low throughput due to spatial limits on the number of trapping sites in a device. Combining methodologies such as hydrophoresis and DEP may provide advantages over those of either technique alone

The DEP device disclosed herein combines hydrophoretic and DEP modules to create a continuous cell sorter that overcomes the limited throughput of DEP trapping devices. The hydrophoretic module directs all cells to the outer edges of the microfluidic channel. This positions cells for separation by the DEP module, in which the induced DEP force directs targeted cells to the middle of the channel. Channel outlets separately collect two cell populations, those remaining along the outer edges of the channel and those focused to the middle of the channel. The DEP device disclosed herein provides continuous, rapid, and label-free cell separations that overcome limitations of sorters using a single separation modality.

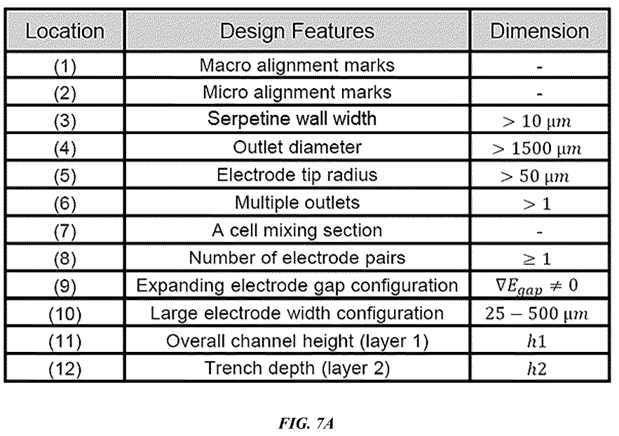

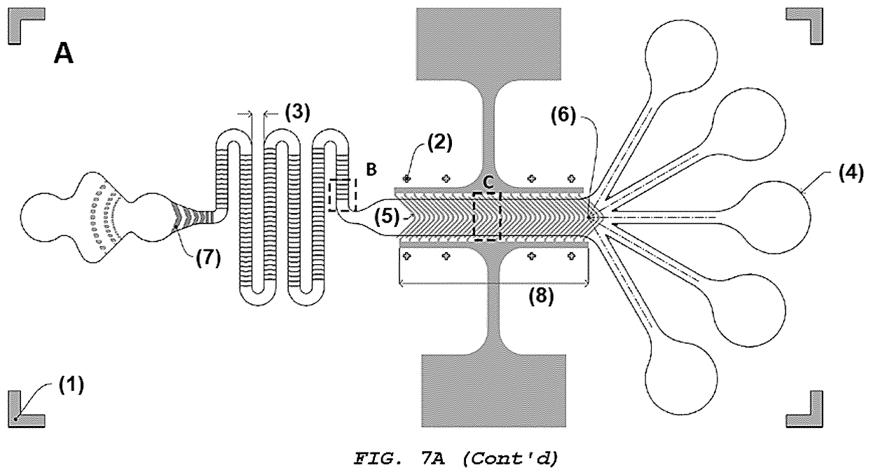

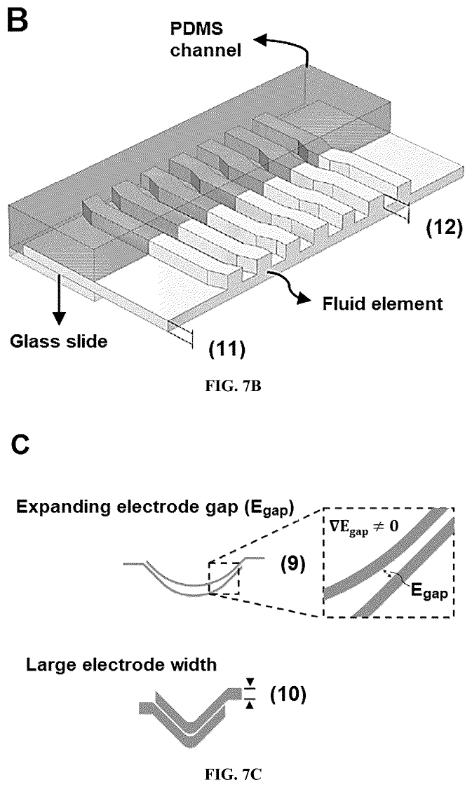

Hydrophoresis is the manipulation of suspended particles using microstructure-induced hydrodynamic pressure gradients. Hydrophoresis can be used to direct cells to specific locations in a microfluidic channel without sheath flow. This simplifies device operation since multiple fluidic inlets with balanced flow rates are not needed to create sheath flow to direct cell position in the channel. The DEP device of the disclosure utilizes a hydrophoretic sheathless aligner working in the laminar flow regime that directs cell location across a wide range of flow rates. This enables efficient and reproducible direction of cells within the channel without costly high-precision instrumentation. The hydrophoretic module pushes cells to the channel edges so that all cells would be at a similar position in the channel when encountering the DEP module. In a particular embodiment, the hydrophoresis module of the DEP device of disclosure comprises a serpentine channel with ridges and trenches to generate a diverging fluid flow that focuses cells into two streams along the channel edges (see FIG. 7A).

Dielectrophoresis (DEP) is a phenomenon in which a force is exerted on a dielectric particle when it is subjected to a non-uniform electric field. This force does not require the particle to be charged. All particles exhibit dielectrophoretic activity in the presence of electric fields. However, the strength of the force depends strongly on the medium and particles' electrical properties, on the particles' shape and size, as well as on the frequency of the electric field. Consequently, fields of a particular frequency can manipulate particles with great selectivity. This has allowed, for example, the separation of cells or the orientation and manipulation of nanoparticles and nanowires. Furthermore, a study of the change in DEP force as a function of frequency can allow the electrical (or electrophysiological in the case of cells) properties of the particle to be elucidated.

In a particular embodiment, the DEP device of the disclosure comprises a DEP module with angled planar interdigitated electrodes in a chevron pattern (see FIG. 7A). The foregoing chevron pattern was designed to pull cells experiencing strong pDEP to the center of the channel, where they would exit via the inner channel outlet. Cells not in pDEP or weak pDEP would remain at the channel edges and exit through the outer channel outlets. The high electric field regions are typically along the electrode edges for planar interdigitated electrodes. Therefore, cells experiencing pDEP feel an induced DEP force perpendicular to the electrodes that pulls the cells toward the electrodes. The pDEP force must be sufficiently strong to attract cells to the electrodes in the presence of the fluid flow. Cells that experience sufficiently strong pDEP to reach the electrodes experience a DEP force perpendicular to the electrode angle. Coupling the induced DEP force with the viscous drag force parallel to the bulk fluid flow causes the cells to migrate along the electrodes and progressively move down the channel toward the outlets. Separation in the dielectrophoresis module is driven by inherent cell electrophysiological properties and does not require cell-type-specific labels. The chevron shape of the electrode array couples with fluid flow in the channel to enable continuous sorting of cells to increase throughput. The integrated system performs better than the trap and release methods used in previous DEP devices by continuously sorting cells, minimizing cell-cell interactions and manual operation, and eliminating residual flow.

The disclosure further provides methods that utilize a DEP device disclosed herein for cell sorting to isolate different populations of cells. For cell sorting using DEP it is important that cells are resuspended in an osmotically balanced, low conductivity buffer. It is also important for post sort applications that cells remain viable in the buffer during the time required for DEP sorting and after plating in normal growth medium.

In a particular embodiment, a method disclosed herein for separating and/or analyzing different populations of cells uses a nonstandard DEP buffer system that improves post-sort acute viability and long-term cell recovery. In the studies presented herein, it was found that a DEP buffer which comprised an agent that promoted and/or stabilized cell to cell contact was highly beneficial for maintaining cancer cell viability and growth. In a particular embodiment the agent is a ROCK inhibitor (ROCKi). ROCK inhibitor (Y27632) inhibits ROCK1 and ROCK2 in the RHO/ROCK pathway.

Sorting to enrich TMZ resistant cells has several advantages over long term growth in TMZ to select resistant clones. Extended growth in TMZ can induce cell characteristics not seen in TMZ resistant cells derived from tumors. For example, D54-TR and U251U251-TR cells were found to be larger than controls, but an association of cell size with TMZ resistance was not observed in acutely sorted cells, suggesting that cell size is not a good indicator of resistance. Secondly, TMZ resistant cells can be rapidly enriched by sorting whereas the process of deriving TMZ resistant cells in culture can take months. Sorted TMZ resistant cells from tumors can be used for molecular characterization and testing of alternative therapeutics, providing a realistically timed pipeline for determining whether different treatment strategies might improve patient outcomes.

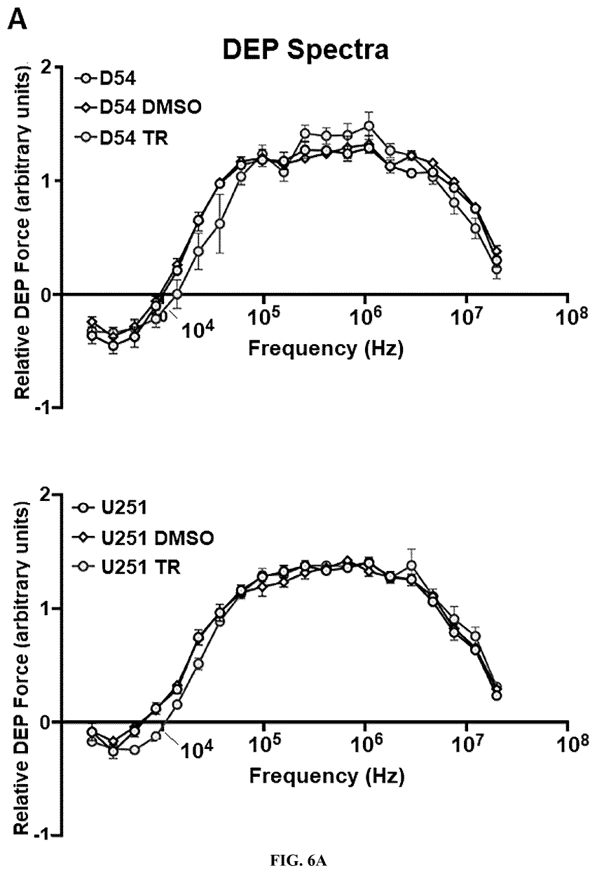

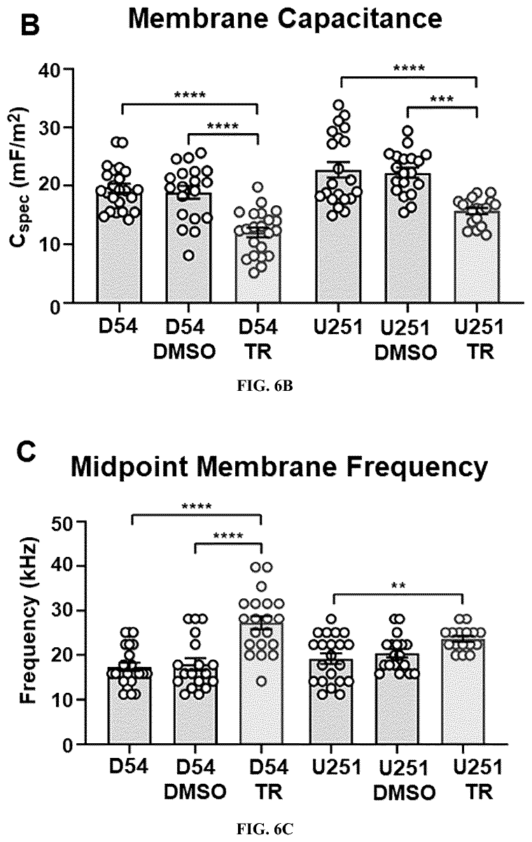

It was tested herein whether TMZ resistant GBM cells could be identified or enriched by comparing the DEP response of TMZ resistant and control cells within a frequency spectrum (see FIG. 6A). It was found herein that GBM cells that varied in TMZ resistance could be identified by the electrophysiological property membrane capacitance, since cells with high TMZ resistance had significantly lower membrane capacitance compared to cells with low TMZ resistance (see FIG. 6B). The midpoint membrane frequency, which predicts whether cells can be effectively sorted by DEP, was higher for TMZ resistant cells compared to controls (see FIG. 6C). To enrich for TMZ resistant GBM cells using DEP, new methods and devices (e.g., DEP devices) were developed.

An embodiment of the DEP device of the disclosure is presented in FIG. 7. As shown in FIG. 7, the structural design features of the DEP device provide for exceptional performance and facile device fabrication in comparison to similar devices in the field. Examples of significant improvements of a DEP device of the disclosure over similar devices in the field include the following features (1)-(13):
(1) Implement macro alignment marks to improve assembly speed. A DEP device disclosed herein is typically constructed with two substrates that are aligned together with micrometer precision. Plasma treatment is often used to treat the two surfaces for bonding; however, the treatment on the surfaces remains effective for only minutes. In a particular embodiment, a DEP device of the disclosure comprises macro alignment marks, which allows for quick course alignment before micro adjustment (see FIG. 7 at 1).
(2) Implement micro alignment marks to improve assembly speed and assignment precision. In a certain embodiment, a DEP device of the disclosure comprises micro alignment marks. After the plasma-treated substrates are coarsely aligned, the micro alignment marks are used for fine alignment (see FIG. 7 at 2).
(3) Increased bonding surface area to increase device robustness. For a DEP device disclosed herein to function properly, the bonded areas need to create a tight seal and remain secure during operation. A positive pressure is used to deliver cells through the microfluidic channel; delamination between the bonded areas could negatively impact performance. The serpentine section of the device has the smallest contact area, which makes it prone to delamination. Accordingly, in a particular embodiment, the walls of the serpentine area have been increased to be >10 μm (see FIG. 7 at 3) so that the contact area has been correspondingly increased, making it far more robust than similar devices in the art.
(4) Increased outlet diameters to increase collection volume, which reduces sample collection frequency. The sorted cells accumulate at the outlets until they are collected by the operator. In a particular embodiment, for a DEP device of the disclosure, the diameter of the outlets is doubled in comparison to similar devices, which increases the volume by four times. As a result, the DEP device of the disclosure can collect four times more cells (see FIG. 7 at 4). Furthermore, the diameter could be further enlarged by elongating the outlet channel length to increase collection volume. Additionally, the outlets could be connected to a collection vessel to collect cells continuously and indefinitely.
(5) Modified electrode tip radius to improve cell release at the electrode tip. At the dielectrophoretic module, cells that experience a strong enough pDEP force are attracted to the electrode, travel toward the center of the fluid channel, and will end up immobilized at the vertex, where the electric field strength is strongest. In a particular embodiment, for a DEP device of the disclosure, the radius of curvature at the electrode tip is increased to 200 μm (see FIG. 7, at 5), which reduces the electric field strength by approximately 40%, releasing the cells.
(6) Implement a design strategy to add more outlets while maintaining fluid pressure among the outlet channels, which enable finer separation into multiple sorted cell fractions. Heterogeneous cell populations are mostly composed of more than two cell subtypes; therefore, the ability to separate them into multiple fractions in a single sort is extremely valuable. In a particular embodiment, the DEP device of the disclosure implements multiple outlets extending from the end of the electrode array radially to create equal pressure among each outlet for continuous and uniform separation (see FIG. 7 at 6).
(7) Implement a cell mixing section before the hydrophoretic alignment section to prolong the consistency of hydrophoretic alignment. The distribution of cells entering the device may be skewed over time, negatively impacting the device's performance. In a particular embodiment, the DEP device of the disclosure provides a cell mixing module that evenly distributes the cells before hydrophoretic alignment (see FIG. 7 at 7). While a hydrophoretic mixing design is shown, other mixing methods, including acoustic actuated mixing, could also be used.
(8) Increased electrode number to increase throughput, reduce the operating voltage, and improve sensitivity. The dielectrophoretic module of the device comprises an array of planar oblique angled electrode arrays that are interdigitally connected, which generates non-uniform electric fields above each electrode pair spanning the entire fluid volume above the electrodes. Cells that move across each electrode pair experience a fluid drag force in the direction of the flow direction and a DEP force normal to the electrode surface. The resulting responses of the cells can be generalized into three groups:
(i) Cells that experience a strong enough positive DEP (pDEP) force will travel along an electrode toward the center of the fluid channel.
(ii) Cells that experience a negative DEP (nDEP) force remain along the sidewalls of the fluid channel.
(iii) Cells that experience an intermediate pDEP force move slightly toward the center of the fluid channel as they move across each electrode.
   For similar devices in the art, to separate Group (iii) from Group (ii), the flow rate needs to be slowed, or the strength of the electric field needs to be increased. In a particular embodiment, for a DEP device of the disclosure, the number of electrode pairs are doubled (see FIG. 7 at 8), which enables better separation of those cells using the same operating parameters or using a faster flow rate (higher throughput) or reduced electric field strength (better cell viability). In further embodiments, additional electrode pairs can be added to improve performance further.
(9) Novel electrode configurations with changing electrode gaps to improve separation resolution. The separation resolution at the dielectrophoretic module can be improved by further dividing the cells into smaller cell fractions that experience the minute difference in DEP force due to differences in their electrophysiological properties. Such separations can be achieved by gradually changing the distance between the electrodes at each electrode pair (see FIG. 7 at 9). For example, EQ. 1 can estimate the induced DEP force on a cell:

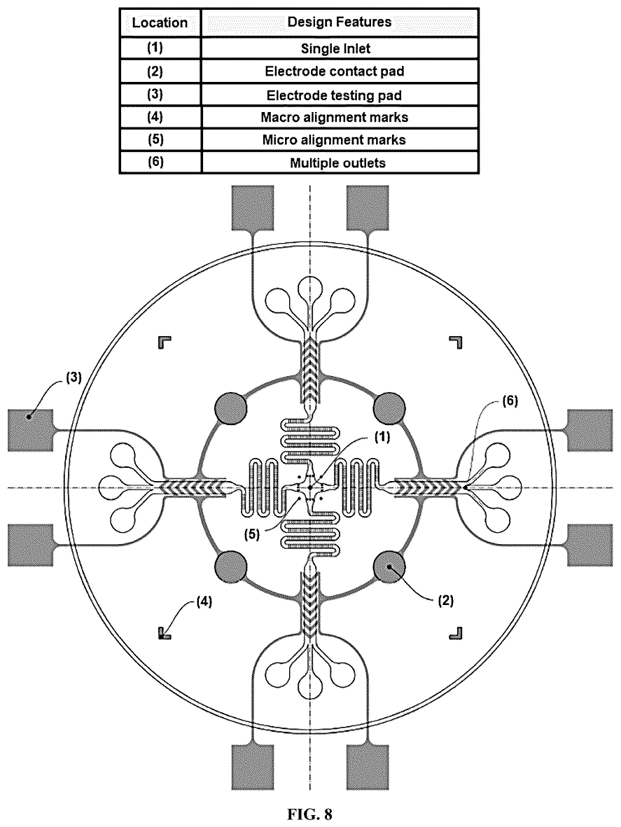

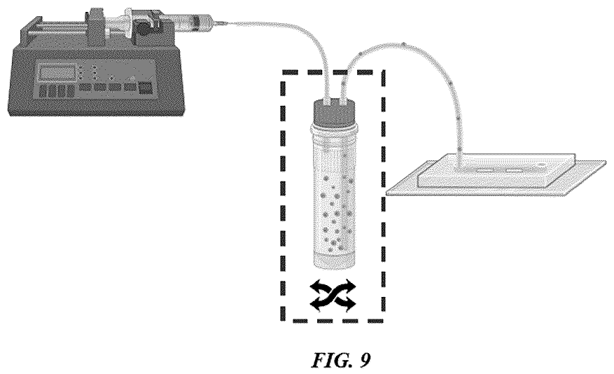

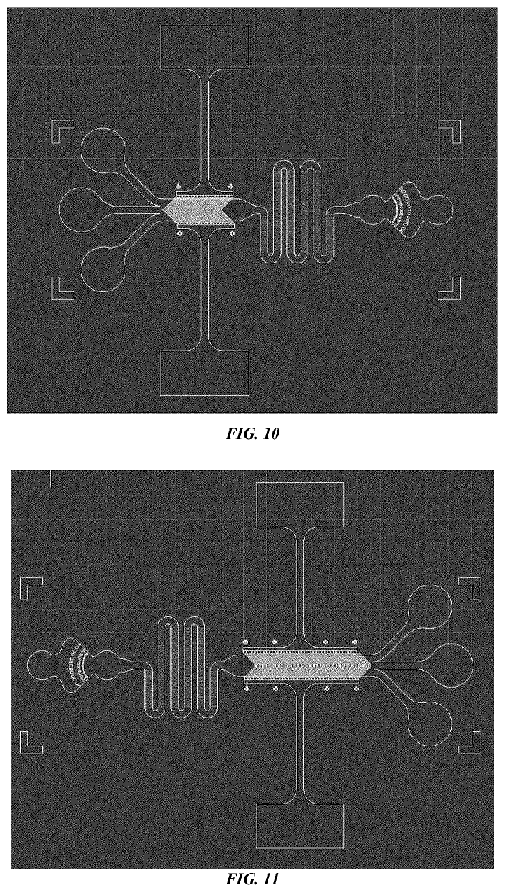

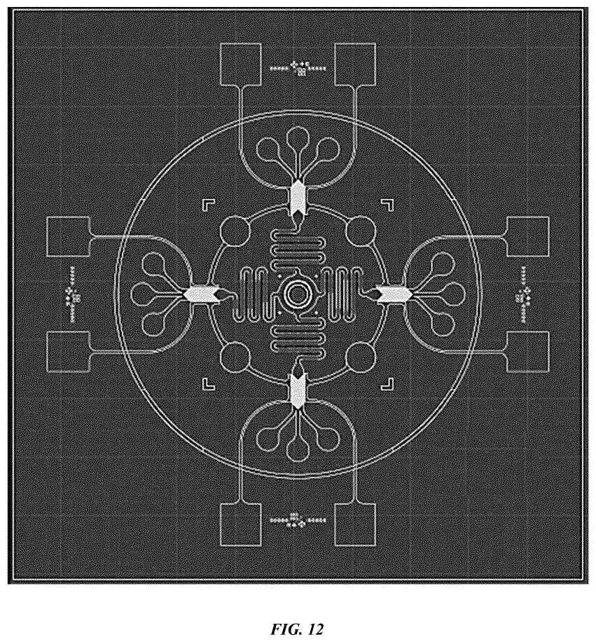

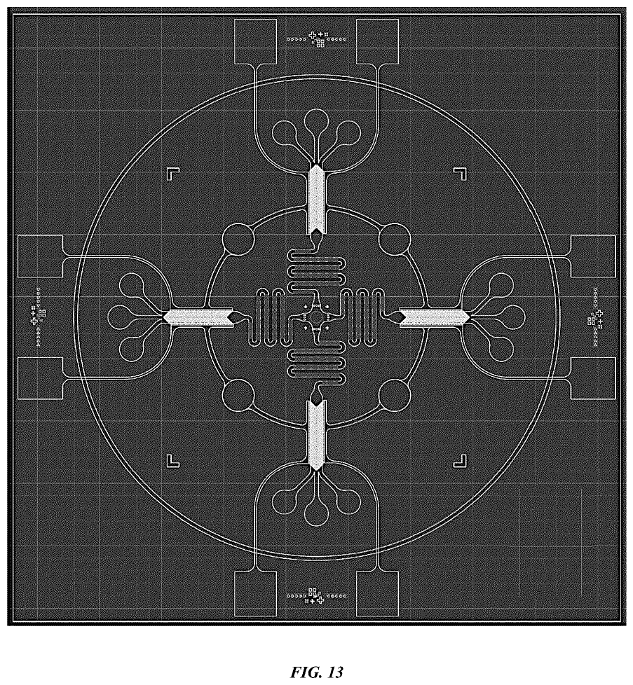

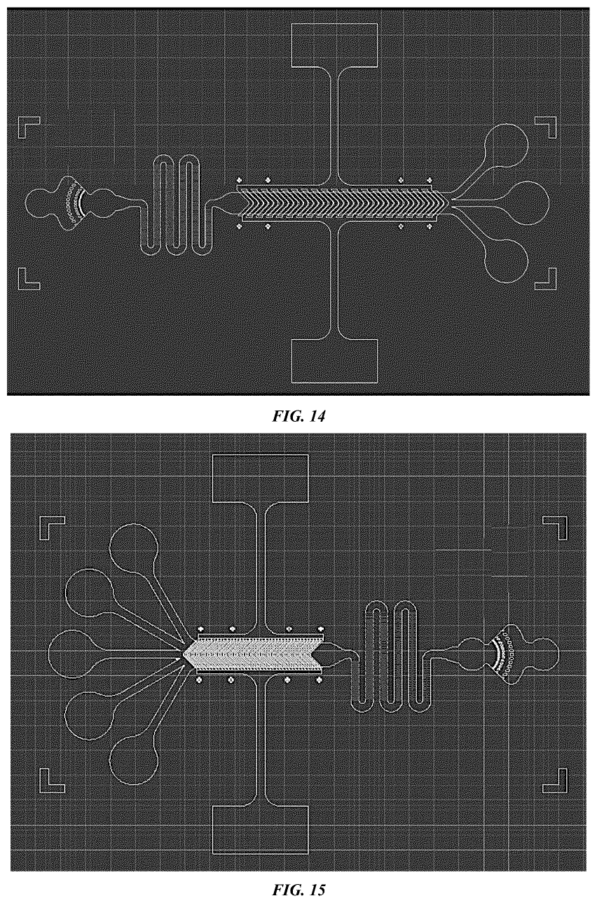

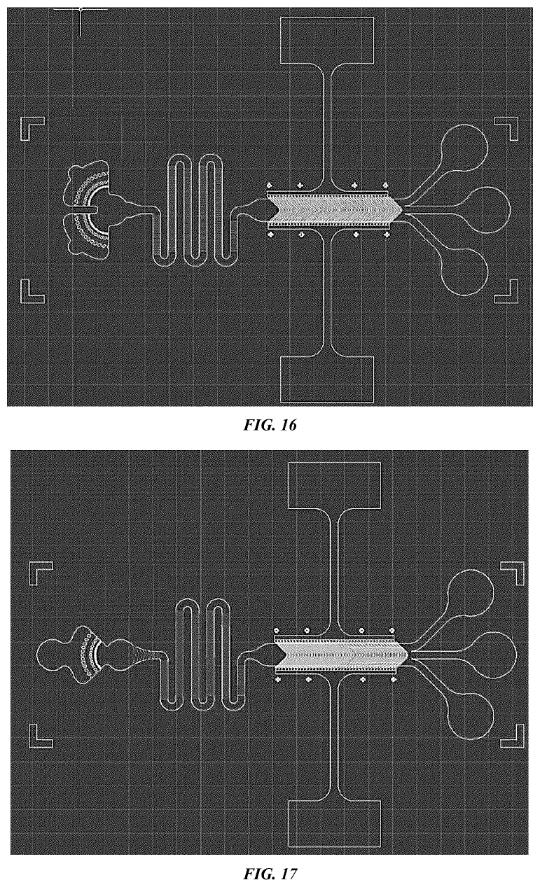

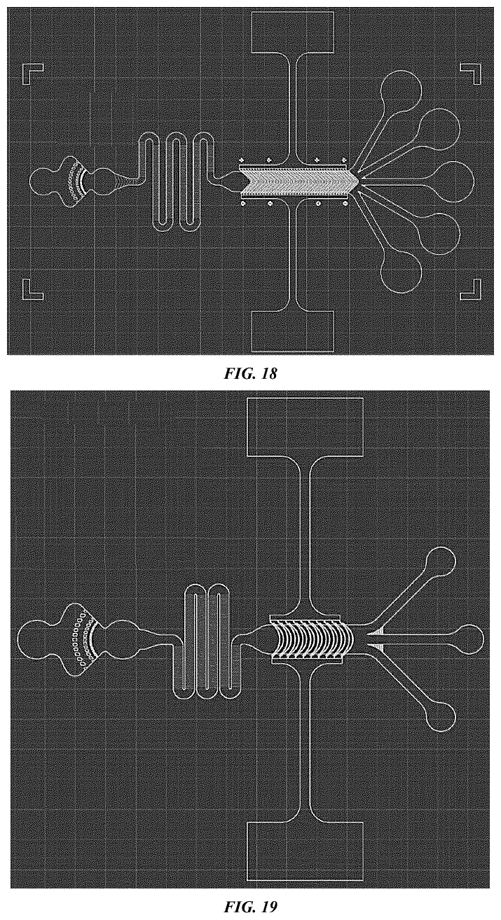

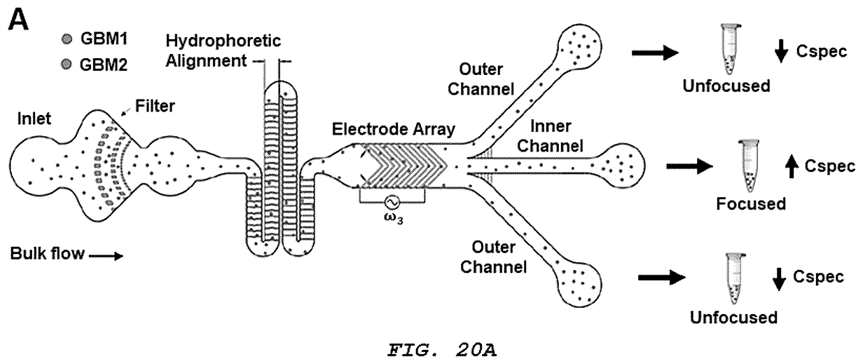

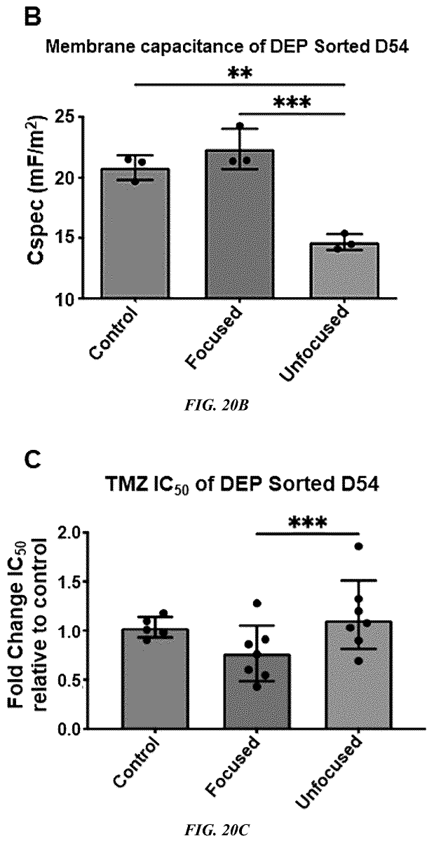

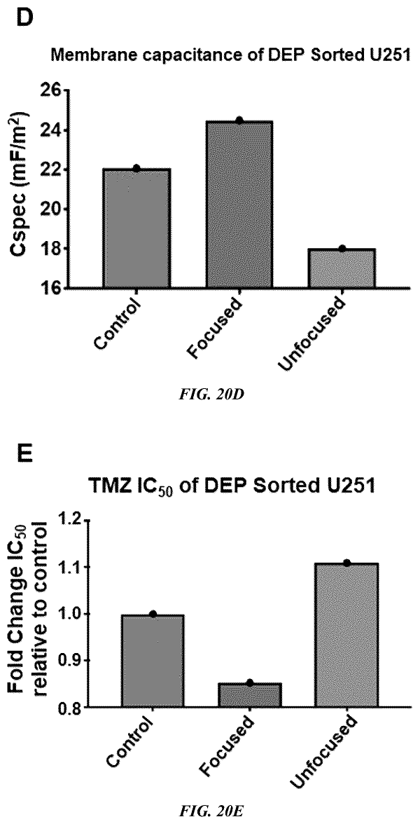

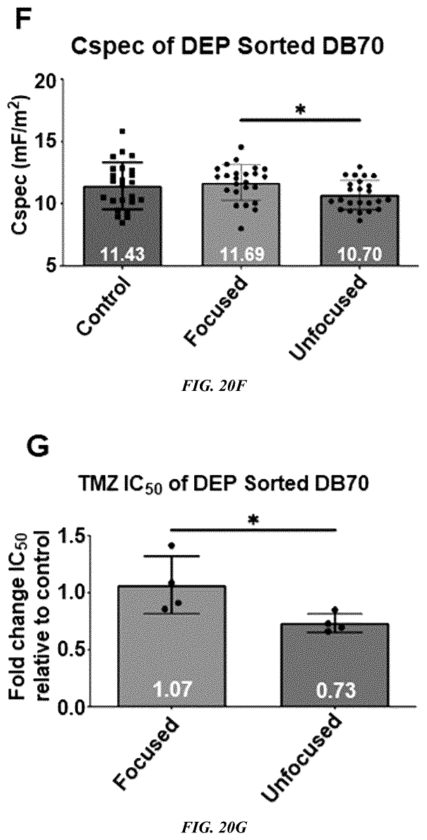

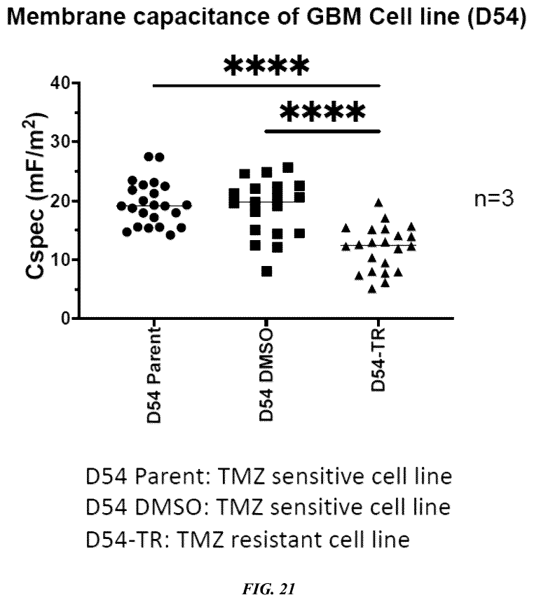

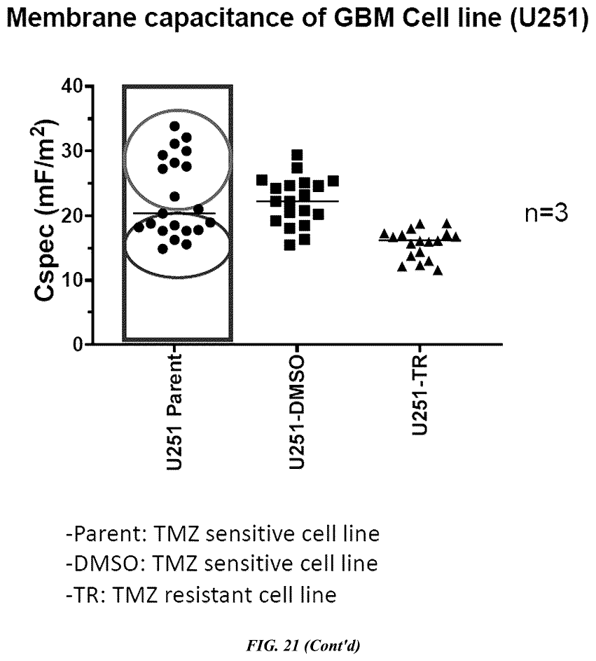

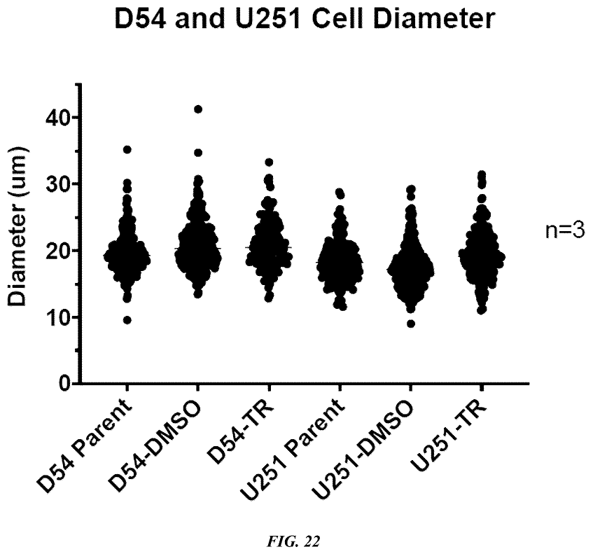

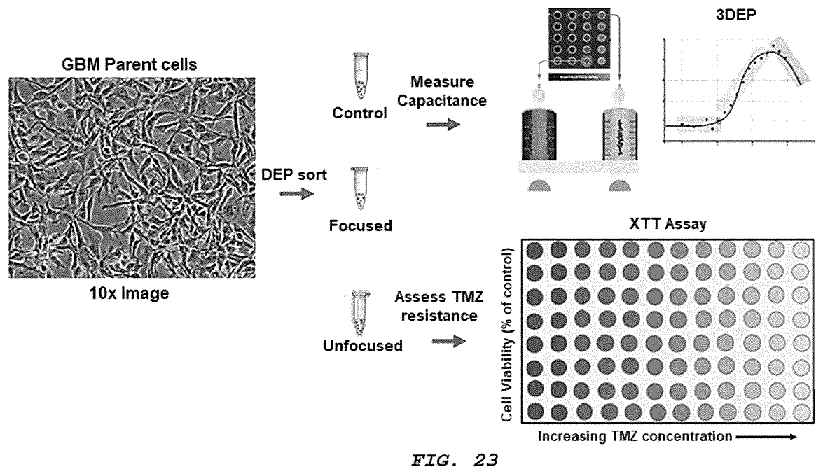

\vec{F}_{DEP}=2πR³ε_{media} Re(CM)∇|\vec{E}|² (1)

where R is the radius of the cell, ε_{media} is the permittivity of the medium, Re(CM) is the real part of the CM factor, and ∇|\vec{E}|² is the gradient of the electric field squared. The electric field can be further simplified into EQ. 2:

$$E = \frac{V}{d} \qquad ( 2 )$$

where V is the applied voltage to the electrode and d is the distance between the electrodes; thus, the DEP force is affected by the gap between the electrodes. The wider the electrode gap, the weaker the DEP force at that electrode region. Hence, cells that differ in their electrophysiological properties will be released at different electrode regions and end up at other outlets. Furthermore, any electrode configurations that change the electrode gap (E_{gap}) may be used, such as different angle electrodes relative to the channel wall.
(10) Increased electrode width to improve DEP focusing force while maintaining cell viability. A Stronger DEP force is desired in most DEP-based systems because it generally allows them to operate at higher speeds. Strong DEP forces are usually achieved by increasing the voltage supply to the electrode; however, too high of a voltage could lead to poor cell viability. Surprisingly, it was found that increasing electrode width will increase DEP-induced movement while limiting the induced transmembrane potential that can harm the cells. FIG. 7 at 10 shows an embodiment of a DEP device of the disclosure with a wider electrode width configuration that exploits this phenomenon.
(11) Novel channel height configuration that reduces the operating voltage to maintain cell viability and promote uniform separation. A uniform separation of the cells at the dielectrophoretic module is achieved when all the cells reach their equilibrium position before leaving the electrode array. However, cells enter this section at various distances above the electrode and cells at the top, experience the weakest DEP force (refer to EQ. 1 and EQ. 2), which might not be able each their equilibrium position. Reducing the channel height at the dielectrophoretic module minimizes this issue. In a particular embodiment, the microfluidic channel of the DEP device of the disclosure is made up of 2 layers (see FIG. 7, at 11 and 12). Typically, channel height at the dielectrophoretic module of DEP-based devices known in the art sums both the heights of each layer making up the module, e.g., summing a first (h1) and a second layer (h2) (i.e., h1+h2). In a particular embodiment, for a DEP device of the disclosure, the overall channel height is modified to equal the height of the first layer (i.e., h1). This configuration reduces the distance between the suspension cells and the electrode, resulting in improved separation efficiency, reduced operating voltage, and improved sensitivity.
(12) The design layout of the significantly improved DEP device of the disclosure integrates multiple sorting units in parallel to improve throughput. Most cell sorting applications require a larger number of sorted cells for downstream applications, such as cell characterization assays, cell transplants, etc. In a particular embodiment, the DEP device of the disclosure integrates multiple sorting units in a radially symmetrical layout and maintaining the single inlet design (see FIG. 8, at 1) to facilitate easy fluidic control and uniform separation. Each side of an interdigital electrode array are connected to the adjacent side of another interdigital electrode array at the electrode contact pads (see FIG. 8, at 2) to simplify electrode actuation. Additional testing pads are connected to every side of the electrode arrays (see FIG. 8, at 3) for quality control, troubleshooting, and provide the flexibility to actuate each sorting units independently. Macro and micro alignment marks are implemented to aid device assembly (see FIG. 8, at 4 and 5). Further, a multiple outlet design is illustrated to enable multiple fraction separation in another embodiment of a DEP device of the disclosure.
(13) Implemented a cell delivery chamber that can maintain consistent cell concentration delivery to the sorting unit(s) over time. The physical density of cells is typically greater than that of the DEP sorting buffer. As a result, cells will settle in their containing vessel and the cell concentration will reduce over time. In attempt to overcome this issue, others have invested in more sophisticated syringe pumps that reorient the syringe vertically so the cells will settle at the tip of the syringe and the cell concentration will increase over time. However, the increase in cell concentration may be undesirable because it could induce cell clogging, and cell-cell interaction during separation, etc. In a particular embodiment, for a DEP device of the disclosure, the cell delivery chamber is a pressurized chamber that is detached from the pump which enables easy mixing of the cell solution in the chamber to ensure more consistent cell concentration (see FIG. 9).

It was further shown herein methods using a DEP device of the disclosure for separating and/or analyzing drug-resistant cancer cells by use of cell intrinsic properties, wherein drug-resistance can be correlated with whole cell membrane capacitance.

The disclosure further provides that the devices, methods and systems described herein can be further defined by the following aspects (aspects 1 to 33):

1. A dielectrophoresis (DEP) device capable of high-throughput continuous dielectrophoretic cell separation or sorting comprising:
one or more inlet channels that can accommodate a fluid input comprising cells;
optionally, one or more filters that are in fluid communication with the one or more inlet channels and one or more hydrophoretic modules, wherein the one or more filters are configured to prevent passage of cell aggregates from the fluid input;
optionally, a cell mixing section in fluid communication with the one or more inlet channels and one or more hydrophoretic modules, wherein the cell mixing section distributes the cells more evenly in the fluid input before flowing into the hydrophoretic module;
one or more hydrophoretic modules that are in fluid communication with the one or more inlet channels and one or more dielectrophoretic modules, wherein the hydrophoretic modules comprise a serpentine channel structure, and wherein the hydrophoretic modules are configured to focus cells into two streams along the edges of the serpentine channel structure;
one or more dielectrophoretic modules comprising an electrode array that are in fluid communication with the hydrophoretic modules and the outlets, wherein the dielectrophoretic modules separate cells by their inherent cell electrophysiological properties, and wherein the dielectrophoretic modules comprise one or more of structural features (i), (ii), (iii) and/or (iv):
   (i) the electrode array comprises 2 or more electrodes;
   (ii) the electrodes having a width from 25 μm to 500 μm;
   (iii) the electrodes having a tip radius of greater than 50 μm; and/or
   (iv) the gap between the electrodes in the electrode array is nonuniform in size;
a plurality of outer outlets in fluid communication with the one or more dielectrophoretic modules that are configured to collect cells that were not focused by DEP; and
one or more inner outlets in fluid communication with the one or more dielectrophoretic module that are configured to collect cells that were focused by DEP;
wherein the plurality of outer outlets and the one or more inner outlets have a diameter that exceeds 1500 μm; and wherein the focused cells of the one or more inner outlets have different dielectric properties than the unfocused cells in the plurality of outer outlets.

2. The DEP device of aspect 1, wherein the DEP device is made from two substrate layers that are aligned and connected or bonded together, particularly, wherein the two substrate layers are irreversibly bonded together.

3. The DEP device of aspect 2, wherein the two substrate layers comprises formable materials that are aligned with or without alignment marks and are connected or bonded together, particularly, wherein the formable materials are selected from chromium, titanium, indium tin oxide (ITO), glass, polydimethylsiloxane (PDMS), polystyrene (PS), polyether ether ketone (PEEK), polyethylene terephthalate (PET), polyvinyl chloride (PVC), polymethylmethacrylate (PMMA), cyclic olefin copolymer (COC), polycarbonate (PC), and polyetherimide (PEI).

4. The DEP device of aspect 3, wherein the formable materials are thermoplastic materials or thermosetting materials, particularly, wherein the thermoplastic material or the thermosetting material is selected from poly dimethylsiloxane (PDMS), polystyrene (PS), polyether ether ketone (PEEK), polyethylene terephthalate (PET), polyvinyl chloride (PVC), polymethylmethacrylate (PMMA), cyclic olefin copolymer (COC), polycarbonate (PC), and polyetherimide (PEI), more particularly, wherein the thermosetting material is poly dimethylsiloxane (PDMS).

5. The DEP device of any one of aspects 2 to 4, wherein at least one of the substrate layers comprises alignment marks to facilitate proper alignment when the two substrates are connected or bonded together, particularly, wherein at least one of the substrates layers comprises two sets of alignment marks, a first set comprising macro-alignment marks which allows for quick orientation of the two substrate layers, and a second set comprising microalignment marks which allows for fine tuning aligning of the two substrates layers.

6. The DEP device of any one of the proceeding aspects, wherein the DEP device further comprises a cell delivery chamber that allows intermittent or continuous mixing of solutions is reversibly attachable to the one or more inlet channels, wherein the cell delivery channel is a pressurized chamber that is reversibly attachable to a pressure exerting device, particularly, wherein the pressure exerting device is a pump, more particularly, wherein the pressure exerting device is a fluidic pump.

7. The DEP device of any one of the proceeding aspects, wherein the DEP device filters comprise one or more filters, and wherein the one or more filters are an array of raised structures that have defined gap sizes between the raised structures, particularly, wherein the raised structures are pillars or columns, more particularly, wherein the one or more filters comprises 2 or 3 series of pillars or columns that have different sized gaps between the pillar or columns, more particularly, wherein the one or more filters comprises 2 or 3 series of pillars or columns wherein the series of pillars columns nearest the hydrophoretic module has the smallest gaps between the pillars or columns, and the series of pillars or columns furthest from the hydrophoretic module has the largest gaps between the pillars or columns.

8. The DEP device of any one of the proceeding aspects, wherein the DEP device comprises the cell mixing section, and wherein the cell mixing section mixes by using hydrophoretic mixing, or acoustic actuated mixing, particularly, wherein the cell mixing section mixes by using hydrophoretic mixing.

9. The DEP device of any one of the proceeding aspects, wherein the walls of the serpentine channel structure of the hydrophoretic modules have a width that is greater than 10 μm, particularly, wherein the hydrophoretic modules comprises gaps that are greater than 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 350 μm, 400 μm, 450 μm, 500 μm, 550 μm, 600 μm, 650 μm, 700 μm, 750 μm, 800 μm, 850 μm, 900 μm, 950 μm, or 1000 μm, or a range that includes or is between any two of the foregoing values.

10. The DEP device of any one of the proceeding aspects, wherein the serpentine channel structure of the hydrophoretic modules comprises microstructures that changes the cross-sectional area of the channel structure to align the cells into two streams along the channel edges, particularly, wherein the microstructures are from 30 μm to 70 μm in height.

11. The DEP device of any one of aspects 2 to 4, wherein at least one of the substrates comprises hydrophoretic features with multiple independent heights, wherein the dielectrophoretic module has a microfluidic channel height that is modified to be less than the overall height of the hydrophoretic features.

12. The DEP device of any one of the proceeding aspects, wherein the dielectrophoretic module comprises structural features (i), (ii), (iii) and (iv).

13. The DEP device of any one of the proceeding aspects, wherein for structural feature (i), the array of electrodes comprises at least 4, 8, 12, 16, 20, 24, 28, 30, 34, 38, 40, 44, 48, 50, 54, 58, 60, 64, 68, 70, 74, 78, 80, 90, 100, 110, 120, 130, 140, 150, 160, 170, 180, 200, 210, 220, 230, 240, 250, 260, 270, 280, 300, 310, 320, 330, 340, 350, 360, 370, 380, 400, 410, 420, 430, 440, 450, 460, 470, 480, or 500 electrodes, or a range of electrodes that includes or is between any two of the foregoing values, particularly, wherein for structural feature (i), the array of electrodes comprises more than 40 electrodes.

14. The DEP device of any one of the proceeding aspects, wherein for structural feature (ii), the width of the electrodes is selected from 50 μm, 55 μm, 60 μm, 65 μm, 70 μm, 75 μm, 80 μm, 85 μm, 90 μm, 95 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 350 μm, 400 μm, and 500 μm, or a range of widths that includes or is between any two of the foregoing values, particularly, wherein for structural feature (i), the array of electrodes comprises from 50 μm to 400 μm.

15. The DEP device of any one of the proceeding aspects, wherein for structural feature (iii), the electrode tip radius is 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, or 300 μm, or a range of radii that includes or is between any two of the foregoing values, particularly, wherein for structural feature (ii), the electrode tip radius is from 100 μm to 250 μm.

16. The DEP device of any one of the proceeding aspects, wherein for structural feature (iv), the gap between the electrodes is variable along the lengths of the electrodes, wherein the gap is narrowest at the base of the electrodes, and most wide at the tip of the electrodes.

17. The DEP device of any one of the proceeding aspects, wherein the DEP device comprises 2 to 4 of outer outlets that are radially orientated from the end of the dielectrophoretic module, particularly, wherein the DEP device comprises 2 or 4 of outer outlets.

18. The DEP device of any one of the proceeding aspects, wherein the diameter of the plurality of outer outlets and the one or more inner outlets are greater than 1500 μm, 1510 μm, 1520 μm, 1530 μm, 1540 μm, 1550 μm, 1560 μm, 1570 μm, 1580 μm, 1590 μm, 1600 μm, 1610 μm, 1620 μm, 1630 μm, 1640 μm, 1650 μm, 1660 μm, 1670 μm, 1680 μm, 1690 μm, 1700 μm, 1710 μm, 1720 μm, 1730 μm, 1740 μm, 1750 μm, 1760 μm, 1770 μm, 1780 μm, 1790 μm, 1800 μm, 1850 μm, 1900 μm, 1950 μm, 2000 μm, 2500 μm, 3000 μm, 3500 μm, 4000 μm, 4500 μm, or 5000 μm, or a range that includes or is between any two of the foregoing diameters, particularly wherein the diameter of the plurality of outer outlets and the one or more inner outlets are greater than 2000 μm.

19. The DEP device of any one of the proceeding aspects, wherein the focused cells of inner outlet have different Cspec values than the unfocused cells in the plurality of outer outlets, particularly, wherein the focused cells of inner outlet have higher Cspec values than the unfocused cells in the plurality of outer outlets.

20. The DEP device of any one of the proceeding aspects, wherein the DEP device comprises one inlet channel, at least 2 hydrophoretic modules; at least 2 dielectrophoretic modules; at least 2 inner outlets; and at least 4 outer outlets.

21. The DEP device of any one of the proceeding aspects, wherein the DEP device comprises one inlet channel; 4 hydrophoretic modules; 4 dielectrophoretic modules; 4 inner outlets; and at least 8 outer outlets.

22. A method to sort or separate a heterogenous population of cells into two separate populations of cells based upon differences in their dielectric properties, the method comprising:
providing a DEP buffer comprising a heterogeneous population of cells into the one or more inlet channels of the DEP device of any one of the preceding aspects;
dissociating the heterogeneous population of cancer cells into single cells in the hydrophoretic modules;
separating the single cells using the one or more dielectrophoretic modules into a focused cell population in the one or more inner output channels and non-focused cell population in the plurality of the outer output channels, particularly wherein the dielectrophoretic modules use alternating and/or direct current.

23. The method of aspect 22, wherein the DEP buffer comprises a ROCK inhibitor.

24. The method of aspect 23, wherein the ROCK inhibitor is Y-27632 or Chroman 1.

25. The method of any one of aspects 22 to 24, wherein the population of heterogeneous cells comprise cancer cells.

26. The method of aspect 25, wherein the cancer cells are derived from a cancer selected from adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, including triple negative breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, Merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, soft tissue sarcoma, uterine cancer, uterine sarcoma, skin cancer (non-melanoma), skin cancer (melanoma), papillomas, actinic keratosis and keratoacanthomas, merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

27. The method of aspect 25, wherein the cancer cells comprise cancer cells that have drug resistance and cancer cells that do not have drug resistance.

28. The method of aspect 27, wherein the drug resistance is resistance to an anticancer agent.

29. The method of aspect 28, wherein the anticancer agent is selected from angiogenesis inhibitors, tyrosine kinase inhibitors, PARP inhibitors, alkylating agents, vinca alkaloids, anthracyclines, antitumor antibiotics, antimetabolites, topoisomerase inhibitors, aromatase inhibitors, mTor inhibitors, retinoids, and HDAC inhibitors.

30. The method of aspect 25, wherein the cancer cells are glioblastoma cancer cells.

31. The method of aspect 30, wherein a portion of the glioblastoma cells have drug resistance, and a portion of the glioblastoma cells do not have drug resistance.

32. The method of aspect 31, wherein the portion of glioblastoma cells that have drug resistance are resistant to a drug selected from temozolomide, bevacizumab, altiratinib, panobinostat, trebanaib, enzastaurin, crenolanib, tandutinib, mibefadil, gliadel, and afatinib.

33. The method of aspect 32, herein the portion of glioblastoma cells have drug resistance to temozolomide.

### Examples

DEP device fabrication. The DEP device is comprised of three main sections: a filter, a sheathless hydrophoretic cell aligner, and a DEP module with oblique parallel electrodes. The channel height is generally uniform, except in the hydrophoretic module where the height varies due to poly dimethylsiloxane (PDMS) microstructures on the channel ceiling. The device has a single inlet directly followed by an array of PDMS posts that create a filter to capture cell clumps. The device comprises macro alignment marks for quick course alignment when the plasma treated PDMS substrate and electrodes are bonded. The device further comprises micro alignment marks that allow for finer alignment after the plasma treated substrates are coarsely aligned using the macro alignment marks.

The structure of the microchannels is created with two-step photolithography. In the first step, a layer of SU-8 2025 photoresist (MicroChem Corp., Newton, MA, USA) is spin coated onto a silicon substrate, and the first layer photomask is manually aligned, and UV cured. In the second step, a second layer of photoresist is spin coated onto the first layer of photoresist, and a second photomask is aligned to the first layer and cured using a mask aligner. Inlet and outlets are punched in the PDMS using a 1.5 mm diameter biopsy punch. The electrodes are fabricated using standard photolithography techniques. Briefly, 200 Å titanium followed by 1000 Å gold are coated on standard 25×75 mm² glass slides using electron-beam physical vapor deposition. The electrode features are transferred onto the gold-coated slide using a Shipley 1827 positive photoresist (Shipley Company, Marlborough, MA, USA).

To assemble the device, the PDMS substrate and the electrode slide are irreversibly bonded after a two-minute oxygen plasma treatment, during which the PDMS substrate and electrode slide are coarsely aligned using the macro alignment marks, followed by finer alignment using the micros alignment marks. Finally, 22-gauge solid copper wires were soldered onto the electrode pads for electrical connection.

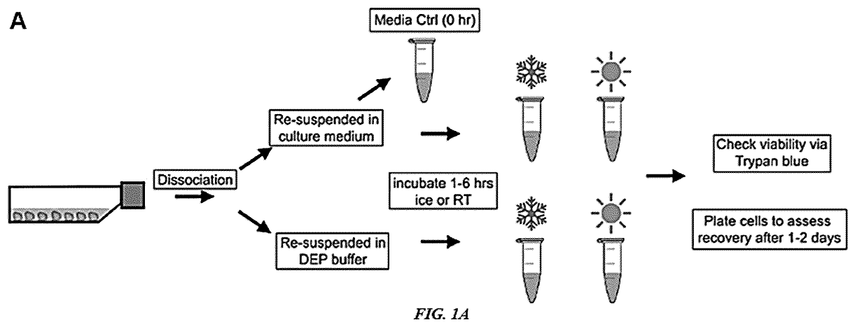

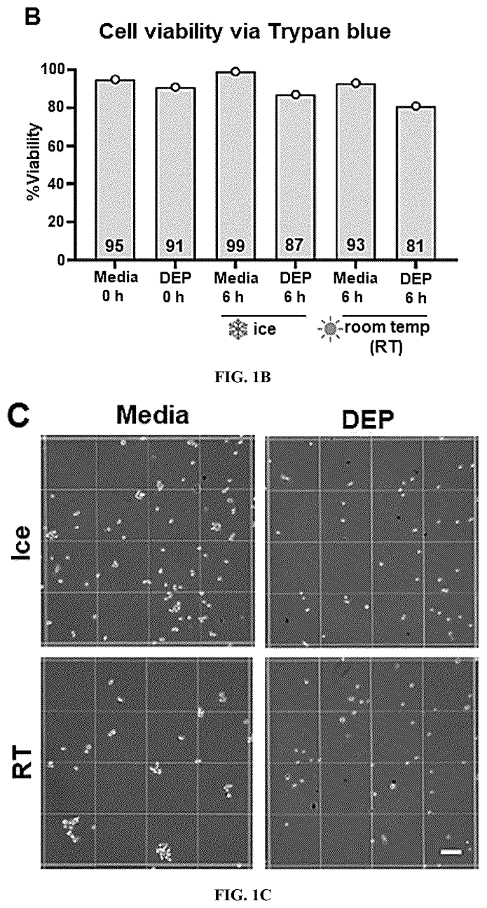

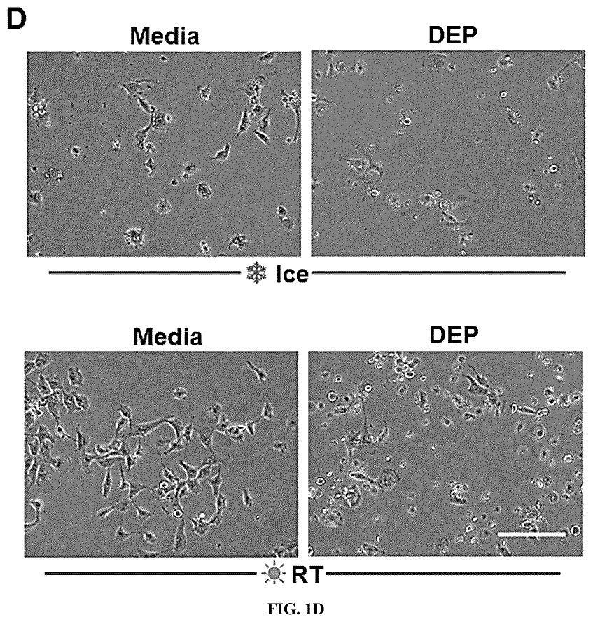

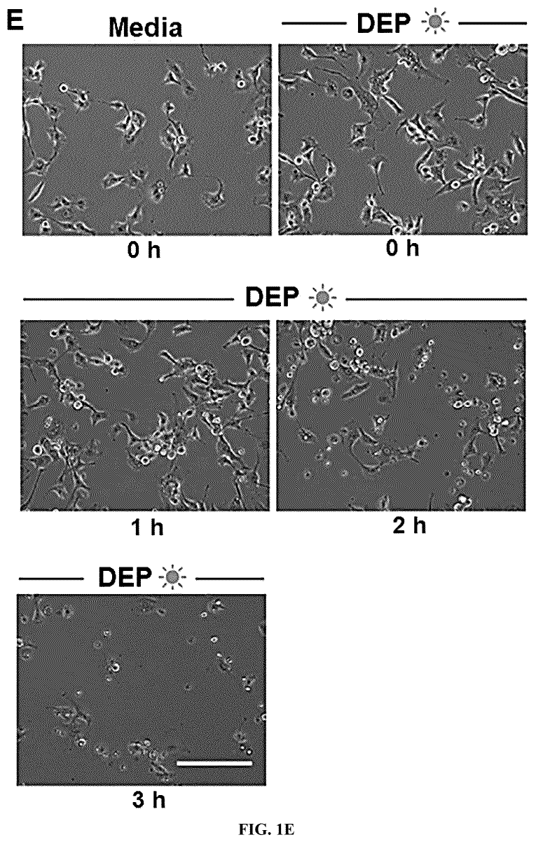

Assessing the viability of D54 cells in a DEP buffer used for sorting mouse and human neural stem and progenitor cells. Cells were incubated in the buffer for up to 6 hours, since this is the maximum time that cells would be in buffer during sorting. Further, the effect of temperature of the buffer (ice or room temperature) on cell viability was also evaluated. All samples showed high viability (over 80%) after incubation, showing that the DEP buffer did not acutely impact cell viability (see FIGS. 1A and C). After the 6-hour buffer incubation, the cells were plated under normal growth conditions and assessed by phase contrast imaging 1-2 days later to assess cell survival and growth. Surprisingly, it was found that the D54 cells incubated in DEP buffer did not recover well and images showed reduced numbers of cells and higher cell death compared to controls (see FIG. 1D). In direct contrast, neural stem and progenitor cells were highly viable when using the same buffer under similar conditions.

Time course study to assess whether the DEP buffer incubation time affected D54 cell survivability. It was found that the cell number was reduced, and greater cell death occurred after 2 hours of incubation in the buffer (see FIG. 1E). It was noted that cells incubated in media at room temperature for 6 hours had the highest number of viable cells and no evidence of cell death after replating and growth (see FIG. 1D). Interestingly, images of these cells immediately after buffer incubation showed clumps of cells, suggesting that cell to cell contact may improve cell survival (see FIG. 1C).

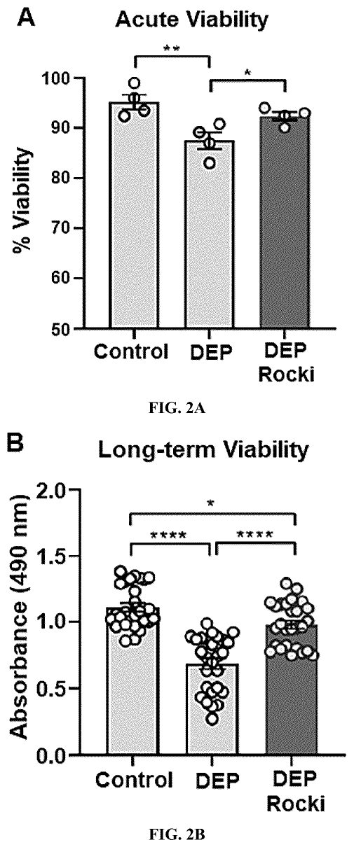

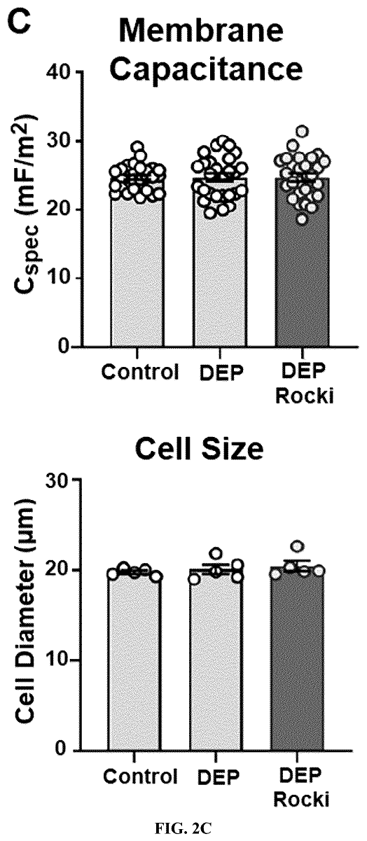

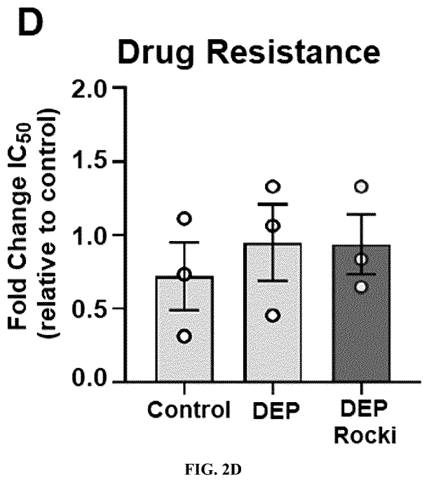

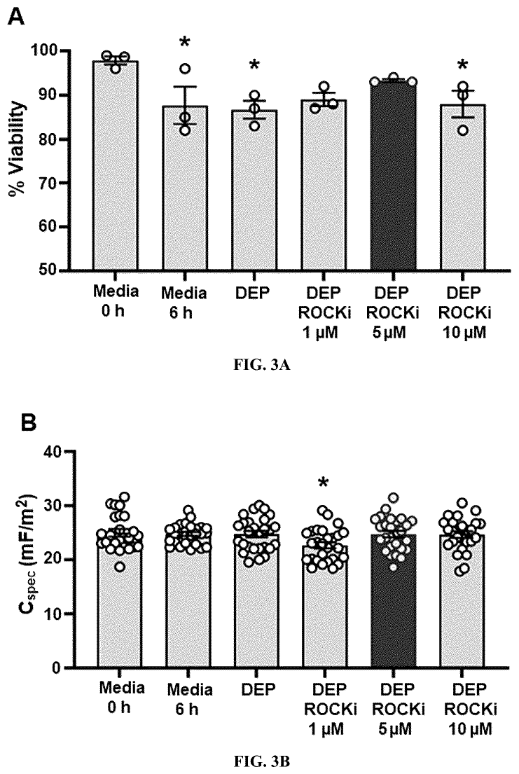

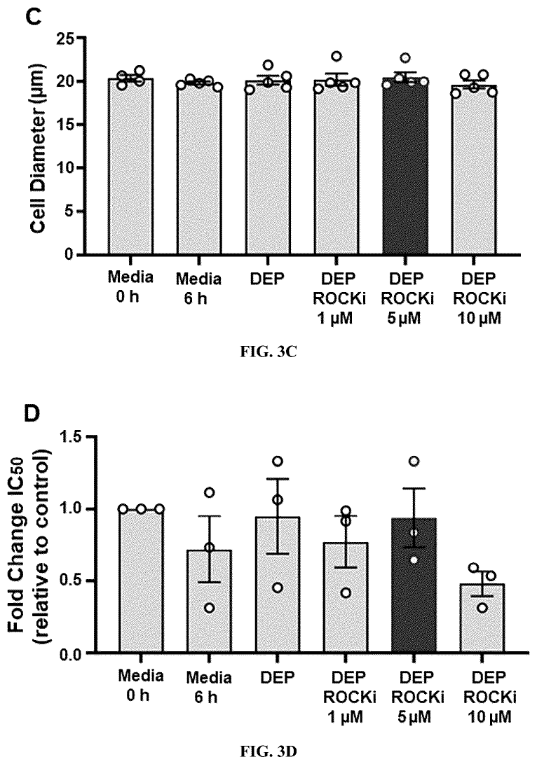

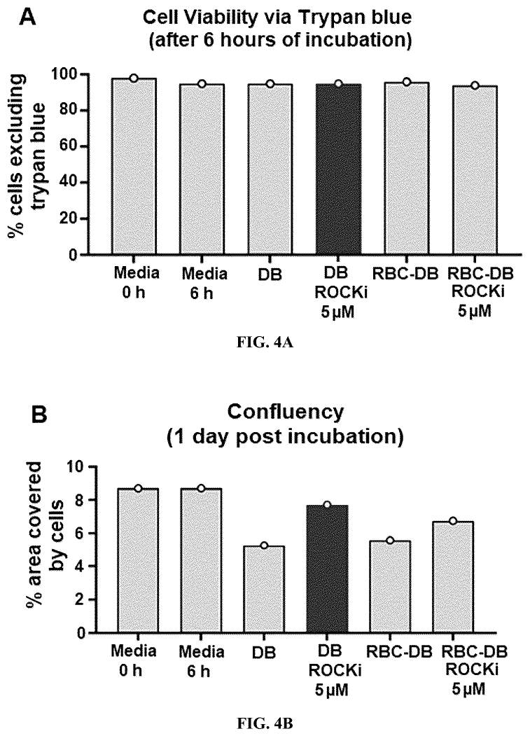

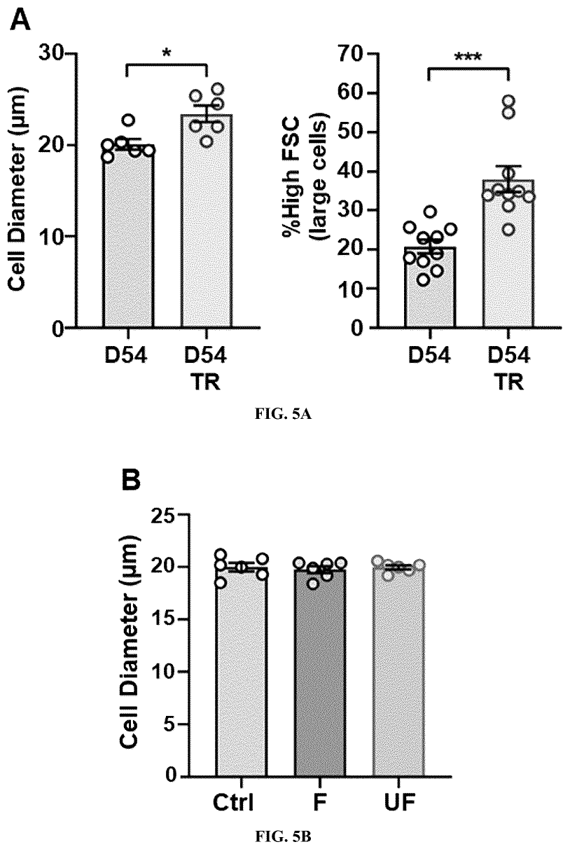

D54 cell viability studies conducted with a modified DEP buffer that included ROCKi. ROCK inhibitor (ROCKi) was included in the DEP buffer at concentrations ranging from 1-10 μM. 5 μM ROCKi was found to be the best concentration for D54 cell viability (see FIGS. 2 and 3). Addition of 5 μM ROCKi to the DEP buffer improved D54 acute viability, as assessed by trypan blue staining, and led to greater than 90% viable cells (see FIG. 2A). An XTT assay was used to measure cell numbers after plating and growth for 2 days. It was found that addition of 5 μM ROCKi to the DEP buffer significantly increased cell numbers compared to DEP buffer without ROCKi (see FIG. 2B). An alternative DEP buffer comprising PBS with calcium and magnesium (0.1 mM Ca and 0.25 mM Mg) was also tested, but this buffer (with or without 5 μM ROCKiR) did not provide higher D54 viability compared to DEP buffer with 5 μM ROCKi (see FIG. 4).

Establishing whether DEP buffer containing affected cell sorting using DEP. Membrane electrophysiological properties (membrane capacitance) and cell size were measured since these directly affect cell responses to an electric field. The addition of 5 μM ROCKi to the DEP buffer did not change the membrane capacitance values or size of D54 cells (see FIGS. 2C and 3).

Testing whether the presence of ROCKi affected the sensitivity of D54 cells to TMZ. ROCKi was included in the DEP buffer, but the cells were plated in normal growth medium without ROCKi for the TMZ assays (as would occur after cell sorting). The presence of 5 μM ROCKi in the DEP buffer did not alter TMZ resistance (see FIGS. 2D and 3). Accordingly, it was found that 5 μM ROCKi can be added to DEP buffer for sorting of D54 cells to improve cell health without altering other cell parameters that would negatively impact sorting.

The use DEP buffer supplemented with 5 uM of ROCK inhibitor greatly improved the sorting of TMZ-resistant GBM cells using the DEP device of the disclosure. Using the novel buffer, D54 parent cells were sorted into a focused and an unfocused cell fraction. The focused cell fraction exhibited a higher Cspec than the unfocused cell fraction. The unfocused cell fraction with lower Cspec was found to be significantly more resistant to TMZ, as indicated by a higher relative IC₅₀ value. The results demonstrated that a DEP buffer supplemented with 5 uM of ROCK inhibitor improve post-sort cell recovery which further enabled post-sort characterization that determined that TMZ resistance cells can be sorted from TMZ susceptible cells based on a difference in Cspec by using a DEP device of the disclosure.

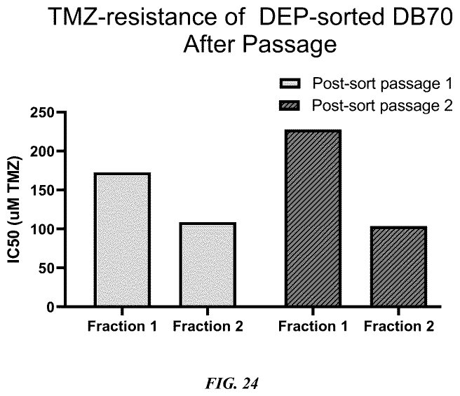

Evaluating whether GBM cells sorted for TMZ-resistant cells, maintained enrichment and resistance over passaging post sorting. Chemotherapeutic resistant cells sorted by a DEP device of the disclosure were cultured for multiple passages (at least 3) after sorting, encompassing at least 10 days for D54 and U251 GBM cells or at least 15 days for GBM cells recently derived from patient tumors (DB70 and DB77) (see FIG. 24). The passaged cells-maintained similar levels of chemotherapeutic resistance during the tested time period.

A number of embodiments have been described herein. Nevertheless, it will be understood that various modifications may be made without departing from the spirit and scope of this disclosure. Accordingly, other embodiments are within the scope of the following claims.

What is claimed is:

1. A dielectrophoresis (DEP) device capable of high-throughput continuous dielectrophoretic cell separation or sorting comprising:
one or more inlet channels that can accommodate a fluid input comprising cells;
optionally, one or more filters that are in fluid communication with the one or more inlet channels and one or more hydrophoretic modules, wherein the one or more filters are configured to prevent passage of cell aggregates from the fluid input;
optionally, a cell mixing section in fluid communication with the one or more inlet channels and one or more hydrophoretic modules, wherein the cell mixing section distributes the cells more evenly in the fluid input before flowing into the hydrophoretic module;
one or more hydrophoretic modules that are in fluid communication with the one or more inlet channels and one or more dielectrophoretic modules, wherein the hydrophoretic modules comprise a serpentine channel structure, and wherein the hydrophoretic modules are configured to focus cells into two streams along the edges of the serpentine channel structure;
one or more dielectrophoretic modules comprising an electrode array that are in fluid communication with the hydrophoretic modules and the outlets, wherein the dielectrophoretic modules separate cells by their inherent cell electrophysiological properties, and wherein the dielectrophoretic modules comprise one or more of structural features (i), (ii), (iii) and/or (iv):
(i) the electrode array comprises 2 or more electrodes;
(ii) the electrodes having a width from 25 μm to 500 μm;
(iii) the electrodes having a tip radius of greater than 50 μm; and/or
(iv) the gap between the electrodes in the electrode array is nonuniform in size;
a plurality of outer outlets in fluid communication with the one or more dielectrophoretic modules that are configured to collect cells that were not focused by DEP; and
one or more inner outlets in fluid communication with the one or more dielectrophoretic module that are configured to collect cells that were focused by DEP;
wherein the plurality of outer outlets and the one or more inner outlets have a diameter that exceeds 1500 μm; and wherein the focused cells of the one or more inner outlets have different dielectric properties than the unfocused cells in the plurality of outer outlets.

2. The DEP device of claim 1, wherein the DEP device is made from two substrate layers that comprise formable materials which are aligned and connected or bonded together.

3. The DEP device of claim 2, wherein the formable materials are selected from gold, chromium, titanium, indium tin oxide (ITO), glass, poly dimethylsiloxane (PDMS), polystyrene (PS), polyether ether ketone (PEEK), polyethylene terephthalate (PET), polyvinyl chloride (PVC), polymethylmethacrylate (PMMA), cyclic olefin copolymer (COC), polycarbonate (PC), and polyetherimide (PEI).

4. The DEP device of claim 2, wherein at least one of the substrate layers comprises hydrophoretic features with multiple independent heights, wherein the dielectrophoretic module has a microfluidic channel height that is modified to be less than the overall height of the hydrophoretic features.

5. The DEP device of claim 1, wherein the DEP device further comprises a cell delivery chamber that allows intermittent or continuous mixing of solutions and is reversibly attachable to the one or more inlet channels, wherein the cell delivery channel is a pressurized chamber that is reversibly attachable to a pressure exerting device.

6. The DEP device of claim 1, wherein the DEP device comprises one or more filters, and wherein the one or more filters are an array of raised structures that have defined gap sizes between the raised structures.

7. The DEP device of claim 1, wherein the DEP device comprises the cell mixing section, and wherein the cell mixing section mixes by using hydrophoretic mixing, or acoustic actuated mixing.

8. The DEP device of claim 1, wherein the walls of the serpentine channel structure of the hydrophoretic modules have a width greater than 10 μm.

9. The DEP device of claim 1, wherein the serpentine channel structure of the hydrophoretic modules comprises microstructures that changes the cross-sectional area of the channel structure to align the cells into two streams along the channel edges.

10. The DEP device of claim 1, wherein for structural feature (ii), the width of the electrodes is from 50 μm to 400 μm.

11. The DEP device of claim 1, wherein for structural feature (iii), the electrode tip radius is from 100 μm to 250 μm.

12. The DEP device of claim 1, wherein for structural feature (iv), the gap between the electrodes is variable along the lengths of the electrodes, wherein the gap is narrowest at the base of the electrodes, and most wide at the tip of the electrodes.

13. The DEP device of claim 1, wherein the DEP device comprises one inlet channel, at least 2 hydrophoretic modules; at least 2 dielectrophoretic modules; at least 2 inner outlets; and at least 4 outer outlets.

14. A method to sort or separate a heterogenous population of cells into two separate populations of cells based upon differences in their dielectric properties, the method comprising:
providing a DEP buffer comprising a heterogeneous population of cells into the one or more inlet channels of the DEP device of claim 1;
dissociating the heterogeneous population of cancer cells into single cells in the hydrophoretic modules; and
separating the single cells using the one or more dielectrophoretic modules into a focused cell population in the one or more inner output channels and non-focused cell population in the plurality of the outer output channels.

15. The method of claim 14, wherein the DEP buffer comprises a ROCK-pathway inhibitor.

16. The method of claim 15, wherein the ROCK-pathway inhibitor is Y-27632 or Chroman 1.

17. The method of claim 14, wherein the heterogeneous population of cells comprise cancer cells.

18. The method of claim 17, wherein the cancer cells are derived from a cancer selected from glioblastoma, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, including triple negative breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, Merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, soft tissue sarcoma, uterine cancer, uterine sarcoma, skin cancer (non-melanoma), skin cancer (melanoma), papillomas, actinic keratosis and keratoacanthomas, merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

19. The method of claim 17, wherein the cancer cells comprise cancer cells that have drug resistance to an anticancer agent and cancer cells that do not have drug resistance to the anticancer agent.

20. The method of claim 19, wherein the anticancer agent is selected from angiogenesis inhibitors, tyrosine kinase inhibitors, PARP inhibitors, alkylating agents, vinca alkaloids, anthracyclines, antitumor antibiotics, antimetabolites, topoisomerase inhibitors, aromatase inhibitors, mTor inhibitors, retinoids, and HDAC inhibitors.
